SEQUENCE LISTING (22 sequences)

SEQ ID NO: 1 - DNA, 3006 bp, Artificial Sequence
  vector genome CB7.CI.HPRT.RBG
  repeat_region (1)..(130): 5′ ITR
  promoter (198)..(863): CB7 hybrid promoter
  misc_feature (198)..(579): CMV IE enhancer
  misc_feature (582)..(863): CB promoter
  TATA_signal (836)..(839): TATA
  Intron (958)..(1930): chicken beta-actin intron
  CDS (1942)..(2601): HPRT
  polyA_signal (2662)..(2788): Rabbit beta globin polyA
  repeat_region (2877)..(3006): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggccacaagatctcccggcgtggtcatcagcgacgacgagcctggctacgacctggacctgttctgcatccccaatcactacgccgaggacctggaacgggtgttcattcctcacggcctgatcatggaccggaccgaaagactggcccgggacgtgatgaaggaaatgggcggacaccacatcgtggccctgtgtgttctgaaaggcggctacaagttcttcgccgacctgctggactacatcaaggccctgaaccggaacagcgatcggagcatccctatgaccgtggacttcatcaggctgaagtcctactgcaacgaccagagcaccggcgacatcaaagtgatcggcggcgacgatctgagcaccctgacaggcaagaacgtgctgatcgtggaagatatcatcgacaccggcaagaccatgcagaccctgctgtctctcgtgcggcagtacaaccccaagatggtcaaggtggccagcctgctggtcaagagaacccctagaagcgtgggctacaagcccgacttcgtgggcttcgagatccccgacaagttcgtcgtgggctacgccctggattacaacgagtacttccgggacctgaaccacgtgtgcgtgatcagcgaaacaggcaaggccaagtacaaggcctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - protein, 218 aa, Artificial Sequence
  Synthetic Construct
MATRSPGVVISDDEPGYDLDLFCIPNHYAEDLERVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVRQYNPKMVKVASLLVKRTPRSVGYKPDFVGFEIPDKFVVGYALDYNEYFRDLNHVCVISETGKAKYKA

SEQ ID NO: 3 - DNA, 660 bp, Artificial Sequence
  engineered nucleic acid sequence HPRT
atggccacaagatctcccggcgtggtcatcagcgacgacgagcctggctacgacctggacctgttctgcatccccaatcactacgccgaggacctggaacgggtgttcattcctcacggcctgatcatggaccggaccgaaagactggcccgggacgtgatgaaggaaatgggcggacaccacatcgtggccctgtgtgttctgaaaggcggctacaagttcttcgccgacctgctggactacatcaaggccctgaaccggaacagcgatcggagcatccctatgaccgtggacttcatcaggctgaagtcctactgcaacgaccagagcaccggcgacatcaaagtgatcggcggcgacgatctgagcaccctgacaggcaagaacgtgctgatcgtggaagatatcatcgacaccggcaagaccatgcagaccctgctgtctctcgtgcggcagtacaaccccaagatggtcaaggtggccagcctgctggtcaagagaacccctagaagcgtgggctacaagcccgacttcgtgggcttcgagatccccgacaagttcgtcgtgggctacgccctggattacaacgagtacttccgggacctgaaccacgtgtgcgtgatcagcgaaacaggcaaggccaagtacaaggcctgatga

SEQ ID NO: 4 - protein, 218 aa, Artificial Sequence; identical to SEQ ID NO: 2
  amino acid sequence HPRT

SEQ ID NO: 5 - DNA, 2229 bp, adeno-associated virus PHP.eB
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 6 - protein, 743 aa, adeno-associated virus PHP.eB
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 2211 bp, adeno-associated virus hu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 8 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - DNA, 2211 bp, adeno-associated virus hu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 10 - DNA, 666 bp, Artificial Sequence
  CB7 hybrid promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 11 - DNA, 973 bp, Artificial Sequence
  chicken beta actin intron
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 12 - DNA, 127 bp, Artificial Sequence
  rabbit beta-globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 13 - DNA, 542 bp, Artificial Sequence
  WPRE element (mut)
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 14 - DNA, 2591 bp, Artificial Sequence
  expression cassette CB7.CI.HPRT.RBG
  enhancer (1)..(382): CMV IE enhancer
  promoter (1)..(666): CB7 hybrid promoter
  promoter (385)..(666): CB promoter
  TATA_signal (639)..(642): TATA signal
  Intron (761)..(1733): chicken beta actin intron
  misc_feature (1745)..(2404): HPRT
  polyA_signal (2465)..(2591): Rabbit beta globin polyA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggccacaagatctcccggcgtggtcatcagcgacgacgagcctggctacgacctggacctgttctgcatccccaatcactacgccgaggacctggaacgggtgttcattcctcacggcctgatcatggaccggaccgaaagactggcccgggacgtgatgaaggaaatgggcggacaccacatcgtggccctgtgtgttctgaaaggcggctacaagttcttcgccgacctgctggactacatcaaggccctgaaccggaacagcgatcggagcatccctatgaccgtggacttcatcaggctgaagtcctactgcaacgaccagagcaccggcgacatcaaagtgatcggcggcgacgatctgagcaccctgacaggcaagaacgtgctgatcgtggaagatatcatcgacaccggcaagaccatgcagaccctgctgtctctcgtgcggcagtacaaccccaagatggtcaaggtggccagcctgctggtcaagagaacccctagaagcgtgggctacaagcccgacttcgtgggcttcgagatccccgacaagttcgtcgtgggctacgccctggattacaacgagtacttccgggacctgaaccacgtgtgcgtgatcagcgaaacaggcaaggccaagtacaaggcctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 15 - DNA, 382 bp, Artificial Sequence
  cytomegalovirus immediate early (CMV IE) enhancer
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 16 - DNA, 282 bp, Artificial Sequence
  chicken beta-actin promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 17 - protein, 599 aa, adeno-associated virus hu68 VP2
TAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 534 aa, adeno-associated virus hu68 VP3
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 137 aa, adeno-associated virus hu68 VP1 fragment
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAK

SEQ ID NO: 20 - protein, 202 aa, adeno-associated virus hu68 VP2 fragment
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLT

SEQ ID NO: 21 - DNA, 1800 bp, adeno-associated virus hu68 VP2
acggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 22 - DNA, 1605 bp, adeno-associated virus hu68 VP3
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

(19) United States
(12) Patent Application Publication
(10) Pub. No.: US 2024/0269328 A1
(43) Pub. Date: Aug. 15, 2024

(54) RECOMBINANT ADENO-ASSOCIATED VIRUSES FOR LESCH-NYHAN DISORDERS AND USES THEREOF

(71) Applicant: The Trustees of the University of Pennsylvania, Philadelphia, PA (US)

(72) Inventors: Christian Hinderer, Baltimore, MD (US); Julia Johansson, Baltimore, MD (US); Eileen Workman, Philadelphia, PA (US)

(73) Assignee: The Trustees of the University of Pennsylvania, Philadelphia, PA (US)

(21) Appl. No.: 18/567,629

(22) PCT Filed: Jun. 8, 2022
(86) PCT No.: PCT/US2022/032647

§ 371 Date: Dec. 6, 2023

Related U.S. Application Data
(60) Provisional application No. 63/341,699, filed on May 13, 2022.
(60) Provisional application No. 63/208,280, filed on Jun. 8, 2021.

(51) Int. Cl. A61K 48/00; A61K 9/00; A61K 38/45; A61P 3/00; C12N 15/86

(52) U.S. Cl. CPC A61K 48/0058; A61K 9/0019; A61K 9/0085; A61K 38/45; A61K 48/0075; A61P 3/00; C12N 15/86; C12Y 204/02008; C12N 2750/14143; C12N 2830/42; C12N 2830/50

(57) ABSTRACT

A rAAV for treatment of a disorder associated with a deficiency in hypoxanthine-guanine phosphoribosyltransferase (HPRT) enzyme levels via gene delivery is provided herein. Also provided herein is a method for treating disorder associated with a deficiency in hypoxanthine-guanine phosphoribosyltransferase (HPRT) enzyme levels by direct delivery to dopaminergic neurons. Correction of Lesch-Nyhan disease may be monitored by assessing dopamine metabolism levels and/or imaging for the presence of dopaminergic cell bodies.

### BACKGROUND OF THE INVENTION

Adeno-associated virus (AAV), a member of the Parvovirus family, is a small non-enveloped, icosahedral virus with single-stranded linear DNA (ssDNA) genomes of about 4.7 kilobases (kb) long. The wild-type genome comprises inverted terminal repeats (ITRs) at both ends of the DNA strand, and two open reading frames (ORFs): rep and cap. Rep is composed of four overlapping genes encoding rep proteins required for the AAV life cycle, and cap contains overlapping nucleotide sequences of capsid proteins: VP1, VP2 and VP3, which self-assemble to form a capsid of an icosahedral symmetry.

Recombinant adeno-associated virus (rAAV) vectors derived from the replication defective human parvovirus have been described as suitable vehicles for gene delivery. Typically, functional rep genes and the cap gene are removed from the vector, resulting in a replication-incompetent vector. These functions are provided during the vector production system but absent in the final vector.

To date, there have been several different well-characterized AA Vs isolated from human or non-human primates (NHP). It has been found that AAVs of different serotypes exhibit different transfection efficiencies, and exhibit tropism for different cells or tissues. Typically, an AAV capsid has 60 copies (in total) of the three variable proteins (vps) that are encoded by the cap gene and have overlapping sequences. These include VP1 (87 kDa), VP2 (73 kDa), and VP3 (62 kDa), which are present in a predicted ratio of 1:1:10, respectively. The entire sequence of VP3 is within VP2, and all of VP2 is within VP1.

Lesch-Nyhan syndrome is an X-linked recessive disorder having a prevalence of about 1 in 300,000 people, making it a rare inborn disorder. This syndrome is associated with an error of purine metabolism characterized by the absence or deficiency of hypoxanthine-guanine phosphoribosyltransferase (HPRT) enzymatic activity. In the absence of HPRT, the purines hypoxanthine and guanine present in certain foods are not built into nucleotides. Uric acid levels are abnormally high in people with Lesch-Nyhan syndrome and sodium urate crystals may abnormally accumulate in the joints and kidneys. Lesch-Nyhan syndrome is inherited as an X-linked recessive genetic disorder that most often affects males. The symptoms of Lesch-Nyhan syndrome include impaired kidney function, acute gouty arthritis, and self-mutilating behaviors such as lip and finger biting and/or head banging. Additional symptoms include involuntary muscle movements, and neurological impairment. See, e.g., National Organization for Rare Disorders, accessed at the following web site: rarediseases.org/rare-diseases/lesch-nyban-syndrome/, 6/6/2021.

Current therapies are inadequate to treat moderate and severe forms of the disease.

### SUMMARY OF THE INVENTION

A therapeutic recombinant (r), replication-defective adeno-associated virus (AAV) is provided herein which is useful for treating and/or reducing symptoms associate with Lesch-Nyhan syndrome or a disorder associated with deficiency in hypoxanthine-guanine phosphoribosyltransferase (HPRT) enzyme levels. A recombinant adeno-associated virus (rAAV) comprising an adeno-associated virus (AAV) capsid and packaged therein a vector genome, wherein the vector genome comprises: (a) an AAV 5′ inverted terminal repeat (ITR), (b) an expression cassette comprising a coding sequence for a hypoxanthine-guanine phosphoribosyltransferase (HPRT) having a nucleic acid sequence of SEQ ID NO: 3 or a sequence at least 80% identical to SEQ ID NO: 3 which encodes amino acid sequence of SEQ ID NO: 4, which coding sequence is operably linked to expression control sequences which direct expression of the HPRT, and (c) an AAV 3′ ITR is provided herein. In certain embodiments, the AAV capsid is selected which is suitable in targeting to dopaminergic neurons. In some embodiments, the AAV capsid is capable of targeting cells in substantia nigra (SN) and/or ventral tegmental area (VTA). In some embodiments, the AAV capsid is selected from those comprising Clade F. In certain embodiments, the AAV capsid is AAVhu68.

In certain embodiments, the rAAV comprises a vector genome comprising AAV 5′ ITR, a promoter, an enhancer, an intron, the coding sequence, a polyA signal, and the AAV 3′ ITR. In some embodiments, the promoter is CB7 promoter. In other embodiments, the promoter is a tyrosine hydroxylase (TH) promoter. In certain embodiments, the vector genome comprises a nucleic acid sequence of SEQ ID NO: 14. In certain embodiments, the vector genome comprises a nucleic acid sequence of nucleotide 1 to nucleotide 3006 of SEQ ID NO: 1.

Also provided herein is a composition comprising a pharmaceutically acceptable aqueous liquid and a population of rAAV as measured in genome copies (GC). In certain embodiments, the rAAV or the composition is suitable for treatment of a disorder associated with a deficiency in hypoxanthine-guanine phosphoribosyltransferase (HPRT) enzyme levels. In some embodiments, the rAAV or the composition is suitable for treatment of Lesch-Nyhan disease.

In certain embodiments, the composition is formulated for direct delivery to the dopaminergic neurons of a patient in need thereof. In some embodiments, the delivery comprises injection into the substantia nigra and/or ventral tegmental area.

Also provided herein is method of treating Lesch-Nyhan Disease, wherein method comprises direct delivery of a therapeutic gene to the dopaminergic neurons of a patient in a need thereof. In certain embodiments, the delivery comprises injection into the substantia nigra and/or ventral tegmental area. In certain embodiments, the therapeutic gene is hypoxanthine-guanine phosphoribosyltransferase (HPRT). In some embodiments, the method comprises direct delivery of an rAAV comprising the therapeutic gene.

These and other embodiments of the invention will be apparent from the following detailed description of the invention.

### BRIEF DESCRIPTION OF THE DRAWINGS

FIG. 1 is an illustration of the anatomy of the brain, including the substantia nigra (SN) and dorsal striatum, wherein substantia nigra is targeted for direct delivery in certain embodiments of the invention provided herein (i.e., site for delivering hypoxanthine-guanine phosphoribosyltransferase (HPRT) to cell bodies of dopaminergic neurons in SN). FIG. 1 shows dorsal striatum (1), substantia nigra (2), targeted region for rescue dopamine levels at synaptic terminals in striatum (3), and targeted region for delivering HPRT to cell bodies of dopaminergic neurons in SN (4).

FIGS. 2A and 2B show hypoxanthine-guanine phosphoribosyltransferase (HPRT) expression and activity in vitro. FIG. 2A shows western blot results of HPRT expression levels measured in harvested samples of HEK293 cells transfected with 3 μg of pAAV.CB7.HPRT.RBG plasmid, wherein GFP-transgene (green fluorescent protein) comprising plasmid was used as a transfection control. FIG. 2B shows HPRT activity levels measured from inosine monophosphate (IMP) concentration (pmol/well) after 30 min in transfected HEK293 cells.

FIGS. 3A to 3F show results of microscopy study using AAV9.PHP.eB (or negative control) in targeting dopaminergic neurons in adult mice stained with tyrosine hydroxylase (red) or green fluorescent protein (GFP). FIG. 3A shows results of microscopy study using negative control in adult mice stained with tyrosine hydroxylase (red). FIG. 3B shows results of microscopy study using AAV9.PHP.eB (IV, 3e11 (3×10¹¹ GC)) in targeting dopaminergic neurons in adult mice stained with tyrosine hydroxylase (red). FIG. 3C shows results of microscopy study using negative control in adult mice stained with green fluorescent protein (GFP). FIG. 3D shows results of microscopy study using AAV9.PHP.eB (IV, 3e11 (3×10¹¹ GC)) in targeting dopaminergic neurons in adult mice stained with green fluorescent protein (GFP). FIG. 3E shows merged results of microscopy study using or negative control in adult mice stained with tyrosine hydroxylase (red) or green fluorescent protein (GFP). FIG. 3F shows merged results of microscopy study using AAV9.PHP.eB (IV, 3e11 (3×10¹¹ GC)) in targeting dopaminergic neurons in adult mice stained with tyrosine hydroxylase (red) or green fluorescent protein (GFP).

FIGS. 4A and 4B show restoration of HPRT expression in adult mouse brain following intravenous administration of AAV9.PHP.eB comprising HPRT transgene. FIG. 4A shows restoration of HPRT expression in adult mouse brain following intravenous administration of AAV9.PHP.eB comprising HPRT transgene, in Groups 1 and 2. FIG. 4B shows restoration of HPRT expression in adult mouse brain following intravenous administration of AAV9.PHP.eB comprising HPRT transgene, in Groups 1 and 3.

FIG. 5A shows dopamine levels in adult HPRT knock out mice (KO) treated with AAV-HPRT. Postnatal HPRT expression reversed striatal dopamine deficit in adult HPRT KO mice. FIG. 5B shows quantification of western blot of whole brain probed with HPRT or β-actin antibodies.

FIGS. 6A and 6B shows delivery of AAV-GFP to the SN in mice. FIG. 6A shows coronal sections of mouse brain from control mouse brain stained with tyrosine hydroxylase (TH, red) to highlight the SN. FIG. 6B shows coronal section of mouse brain from AAV-GFP injected mouse brain (GFP, green).

FIGS. 7A and 7B shows intraparenchymal delivery of AAV-HPRT to SN in HPRT KO mice. FIG. 7A shows quantitation of Western blot measuring HPRT expression in the SN. FIG. 7B shows HPLC measurement of dopamine levels in the striatum.

FIGS. 8A to 8C shows MRI-guided intraparenchymal delivery of AAV-HPRT to SN in a nonhuman primate. FIG. 8A shows sagittal view showing needle track (arrow) and infusion site. FIG. 8B shows axial view showing left and right infusion sites (arrows). FIG. 8C shows coronal view showing two infusion sites (arrows).

### DETAILED DESCRIPTION OF THE INVENTION

Provided herein are rAAV vectors expressing hypoxanthine-guanine phosphoribosyltransferase (HPRT) and compositions containing same, optionally formulated for direct injection into the substantia nigra and/or ventral tegmental area. In certain embodiments, methods for direct delivery of a Lesch-Nyhan therapeutic are provided, e.g., injection into the substantia nigra and/or ventral tegmental area. In certain embodiments, methods for monitoring HPRT treatments by assessing brain morphology and dopamine metabolism are provided.

### Recombinant Adeno-Associated Virus

Recombinant adeno-associated virus (rAAV) based compositions and methods for treating Lesch-Nyhan syndrome (also referred to as Lesch-Nyhan disease, Lesch Nyhan syndrome, Lesch Nyhan disease, or LNS), or a disease associated with deficiency in hypoxanthine-guanine phosphoribosyltransferase (HPRT) enzyme levels.

An engineered coding sequence for HPRT is provided herein having the sequence of SEQ ID NO: 3 or a sequence about 80% identical thereto which encodes SEQ ID NO: 4. In certain embodiments, the sequence is about 85% identical, about 90% identical, about 95% identical, about 97% identical, about 98% identical, 99% identical, or values therebetween. Suitably, the engineered HPRT coding sequence is engineered into an expression cassette comprising at least one open reading frame (ORF) and is operably linked to regulatory sequences which direct expression thereof in a target cell, e.g., dopaminergic neurons.

### Expression Cassette and Vector Genome

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3″ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

In certain embodiments, the transgene, e.g., HPRT, may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding HPRT operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed MITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

In one embodiment, the provided herein is a recombinant adeno-associated virus (rAAV) comprising an adeno-associated virus (AAV) capsid and packaged therein a vector genome, wherein the vector genome comprises: (a) an AAV 5′ inverted terminal repeat (ITR), (b) an expression cassette comprising at least one open reading frame (ORF) comprising a coding sequence for a hypoxanthine-guanine phosphoribosyltransferase (HPRT) having a nucleic acid sequence of SEQ ID NO: 3 or a sequence at least 80% identical thereto which encodes amino acid sequence of SEQ ID NO: 4, which coding sequence is operably linked to expression control sequences which direct expression of the HPRT, and (c) an AAV 3′ ITR.

In addition to the major elements identified above for the recombinant AAV vector, the vector also includes conventional control elements (i.e., regulatory sequences) necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. As described herein, regulatory elements comprise but not limited to: promoter: enhancer: transcription factor: transcription terminator: efficient RNA processing signals such as splicing and polyadenylation signals (polyA): sequences that stabilize cytoplasmic mRNA, for example Woodchuck Hepatitis Virus (WHP) Posttranscriptional Regulatory Element (WPRE): sequences that enhance translation efficiency (i.e., Kozak consensus sequence).

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products (e.g., HPRT) include, but are not limited to chicken beta-actin (CB) promoter, CB7 promoter (also referred to as CB7 hybrid promoter comprising a cytomegalovirus immediate early (CMV IE) enhancer, optionally a linker sequence, and a chicken beta-actin promoter), human cytomegalovirus (CMV) promoter, ubiquitin C promoter (UbC), the early and late promoters of simian virus 40 (SV40), U6 promoter, metallothionein promoters, EFlα promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art. In certain embodiments, the CB promoter comprises nucleic acid sequence of SEQ ID NO: 16. In certain embodiments, the CB7 hybrid promoter comprises a CMV IE enhancer comprising nucleic acid sequence of SEQ ID NO: 15, optionally a linker sequence, and a CB promoter comprising nucleic acid sequence of SEQ ID NO: 16. In certain embodiments, the CB7 hybrid promoter comprises nucleic acid sequence of SEQ ID NO: 10.

Examples of tissue- or cell-specific promoters suitable for use in the present invention include, but are not limited to, endothelin-I (ET-I) and Flt-I, which are specific for endothelial cells, FoxJI (that targets ciliated cells). Other examples of tissue specific promoters suitable for use in the present invention include, but are not limited to, liver-specific promoters. In certain embodiments, the promoter is a tissue-specific (e.g., neuron-specific) promoter. In certain embodiments, a suitable promoter may include without limitation, an elongation factor 1 alpha (EF1 alpha) promoter (see, e.g., Kim DW et al, Use of the human elongation factor 1 alpha promoter as a versatile and efficient expression system. Gene. 1990 Jul 16:91(2):217-23), a Synapsin 1 promoter (see, e.g., Kügler S et al, Human synapsin I gene promoter confers a highly neuron-specific long-term transgene expression from an adenoviral vector in the adult rat brain depending on the transduced area. Gene Ther. 2003 Feb: 10(4):337-47), a shorted synapsin promoter, a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 Feb: 145(2):613-9. Epub 2003 Oct 16), or a CB6 promoter (see, e.g., Large-Scale Production of Adeno-Associated Viral Vector Serotype-9 Carrying the Human Survival Motor Neuron Gene, Mol Biotechnol. 2016 Jan:58(1):30-6. doi: 10.1007/s12033-015-9899-5). Preferably, such promoters are of human origin. In certain embodiments, the promoter is human tyrosine hydroxy lase (TH) promoter (THa) (expresses in several cell groups within the brain, including the dopaminergic (DA) neurons of the substantia nigra and ventral tegmental area, and the noradrenergic neurons of the locus coeruleus). See, e.g., Kessler, M, et al., Brain Res Mol Brain Res, 2003, April 10, 112(1-2):8-23: Oh, M. S., et al., Nat Gene Ther, 2009, 16: 437-440).

In one embodiment, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch (R Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551): or a hormone response element such as described by Lee et al. (1981, Nature 294:228-232): Hynes et al. (1981, Proc. Natl. Acad. Sci. USA 78:2038-2042): Klock et al. (1987, Nature 329:734-736); and Israel & Kaufman (1989, Nucl. Acids Res. 17:2589-2604) and other inducible promoters known in the art. These response elements may include, a hypoxia response element (HRE) that binds HIF-Ia and ß, a metal-ion response element such as described by Mayo et al. (1982, Cell 29:99-108): Brinster et al. (1982, Nature 296:39-42) and Searle et al. (1985, Mol. Cell. Biol. 5:1480-1489): or a heat shock response element such as described by Nouer et al. (in: Heat Shock Response, ed. Nouer, L., CRC, Boca Raton, Fla., ppI67-220, 1991).

Using such promoters, expression of the transgene can be controlled, for example, by the Tet-on/off system (Gossen et al., 1995, Science 268: 1766-9; Gossen et al., 1992, Proc. Natl. Acad. Sci. USA., 89(12):5547-51): the TetR-KRAB system (Urrutia R., 2003, Genome Biol., 4(10):231: Deuschle U et al., 1995, Mol Cell Biol. (4): 1907-14): the mifepristone (RU486) regulatable system (Geneswitch: Wang Y et al., 1994, Proc. Natl. Acad. Sci. USA., 91(17):8180-4: Schillinger et al., 2005, Proc. Natl. Acad. Sci. USA.102(39):13789-94); and the humanized tamoxifen-dep regulatable system (Roscilli et al., 2002, Mol. Ther. 6(5):653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally or systemically to the AAV-transfected cells.

In some embodiment, the regulatory elements comprise an enhancer. In some embodiments, suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a cytomegalovirus immediate early (CMV IE) enhancer. This enhancer may be present in two copies which are located adjacent to one another. Alternatively, the dual copies of the enhancer may be separated by one or more sequences. In a further embodiment, the enhancer(s) is selected from one or more of an APB enhancer, an ABPS enhancer, an alpha mic/bik enhancer, a TTR enhancer, an en34 enhancer, an ApoE enhancer, a CMV enhancer, or an RSV enhancer. In yet another embodiment, the regulatory elements comprise an intron. In a further embodiment, the intron is selected from chicken beta actin intron (CBA), human beta globin, IVS2, SV40, bGH, alpha-globulin, beta-globulin, collagen, ovalbumin, or p53. In certain embodiments, the chicken beta actin intron comprises nucleic acid sequence of SEQ ID NO: 11. See, e.g., WO 2011/126808. In one embodiment, the regulatory elements comprise a polyA. In a further embodiment, the polyA is a synthetic polyA or from bovine growth hormone (bGH), human growth hormone (hGH), SV40, rabbit ß-globin (also referenced as rabbit beta-globin or RBG), or modified RGB (mRBG). In certain embodiments, the rabbit beta-globin polyA signal comprises nucleic acid sequence of SEQ ID NO: 12. In another embodiment, the regulatory elements may comprise a WPRE sequence, which may be engineered upstream of the polyA sequence and downstream of the coding sequence. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene. See also, Kingsman S. M., Mitrophanous K., & Olsen J. C. (2005), Potential Oncogene Activity of the Woodchuck Hepatitis Post-Transcriptional Regulatory Element (Wpre).” Gene Ther. 12(1):3-4; and Zanta-Boussif M. A., Charrier S., Brice-Ouzet A., Martin S., Opolon P., Thrasher A. J., Hope T. J., & Galy A. (2009), Validation of a Mutated Pre Sequence Allowing High and Sustained Transgene Expression While Abrogating Why-X Protein Synthesis: Application to the Gene Therapy of Was, Gene Ther. 16(5):605-19, both of which are incorporated herein by reference in its entirety. See also, SEQ ID NO: 13 (WPRE element mutated). In other embodiments, enhancers are selected from a non-viral source. In certain embodiments, no WPRE sequence is present. In yet another embodiment, the regulatory elements comprise a Kozak sequence.

In one embodiment, the expression cassette comprises regulatory elements which direct expression of a sequence encoding one or more elements of a gene replacement system for delivering HPRT. In one embodiment, the regulatory elements comprise one or more promoters. In certain embodiments, the expression cassette includes a constitutive or a regulatable promoter. In certain embodiments, the promoter is a tissue-specific (e.g., dopaminergic neuron specific) promoter.

Optionally, the expression cassette may include miRNA target sequences in the untranslated region(s). The miRNA target sequences are designed to be specifically recognized by miRNA present in cells in which transgene expression is undesirable and/or reduced levels of transgene expression are desired. In certain embodiments, the expression cassette includes miRNA target sequences that specifically reduce expression of the HPRT in dorsal root ganglion (DRG), e.g., to reduce drg toxicity and/or axonopathy. See, e.g., PCT/US2019/67872, filed Dec. 20, 2019 and now published as WO 2020/132455 and PCT/US2021/32003, filed May 12, 2021, now published as WO 2021/231579, all of which are incorporated herein by reference in their entireties. In certain embodiments, the miRNA target sequences are located in the 3′ UTR, 5′ UTR, and/or in both 3′ and 5′ UTR. In some embodiments, the miRNA target sequences are operably linked to the regulatory sequences in the expression cassette. In certain embodiments, the expression cassette comprises at least two tandem repeats of DRG-specific miRNA target sequences, wherein the at least two tandem repeats comprise at least a first miRNA target sequence and at least a second miRNA target sequence which may be the same or different. In certain embodiments, the tandem miRNA target sequences are continuous or are separated by a spacer of 1 to 10 nucleic acids, wherein said spacer is not a miRNA target sequence.

In certain embodiments, an expression cassette comprising a hypoxanthine-guanine phosphoribosyltransferase (HPRT) coding sequence is provided herein, e.g., nucleotide 198 to nucleotide 2788 of SEQ ID NO: 1 (or SEQ ID NO: 14: CB7.CI. HPRT. RBG). In certain embodiments, the expression cassette comprises a CB7 promoter, a chicken beta actin intron, a nucleic acid sequence encoding HPRT (i.e., HPRT coding sequence), and a rabbit beta-globin polyA. In one embodiment, the expression cassette (or a vector genome, nt. 1 to 3006 of SEQ ID NO: 1) comprising same is engineered into a suitable genetic element (e.g., a plasmid) for use in packaging into an AAV capsid. In one embodiment, the expression cassette comprising nucleic acid sequence of SEQ ID NO: 14 is engineered into a suitable genetic element (e.g., a plasmid) for use in packaging into an AAV capsid. The resulting rAAV comprises the vector genome.

It should be understood that the compositions in the expression cassettes described herein are intended to be applied to the compositions and methods described across the specification.

In one embodiment, the vector genome comprises a AAV 5′ ITR, optionally a spacer sequence, a CB7 promoter, a chicken beta-actin intron, a HPRT coding sequences, a rabbit beta globin polyA, optionally a spacer sequences, and a AAV 3′ ITR. In certain embodiments, a plasmid has a vector genome comprising ITRs with a shortened 130 nucleotide sequence, optionally derived from AAV2, which reverts to the full-length 145 nucleotide ITR when it replicates and is packaged into the AAV capsid. In certain embodiments, the vector genome comprises stuffer or spacer sequences between the AAV 5′ ITR and the promoter and/or the polyA and the AAV 3′ ITR. In certain embodiments, a spacer sequence is about 50 to about 100 nucleotides or about 66 nucleotides between the AAV 5′ ITR and the promoter. In certain embodiments, a spacer sequence is about 50 to about 100 nucleotides or about 88 nucleotides between the AAV3′ ITR and the polyA.

In one embodiment, a vector genome encoding HPRT is provided herein, e.g., SEQ ID NO: 1 (AAV2-5′ITR—CB7 promoter—chicken beta actin intron—HPRT coding sequence—rabbit beta-globin polyA—AAV2 3′ITR).

In certain embodiments, provided herein is a recombinant adeno-associated virus (rAAV) useful as CNS-directed therapy for treatment of a subject having Lesch-Nyhan syndrome, wherein the rAAV comprises an AAV capsid, and a vector genome packaged therein comprising an expression cassette comprising at least one open reading frame (ORF) comprising a coding sequence for an HPRT of nucleic acid sequence of SEQ ID NO: 3 or a sequence at least about 80% identical to SEQ ID NO: 3 which encodes SEQ ID NO: 4. In certain embodiments, the rAAV comprises the vector genome comprising: (a) an AAV 5′ inverted terminal repeat (ITR): (b) a sequence encoding HPRT which is operably linked to regulatory elements which direct expression thereof in a host cell: (c) regulatory elements which direct expression; and (d) an AAV 3′ ITR (e.g., rAAV.HPRT). In one embodiment, the rAAV has a tropism for a cell of the CNS (e.g., an rAAV bearing an AAV9.PHP.eB, AAVhu68 capsid or an AAVrh91 capsid), and/or contains a neuron-specific expression control elements (e.g., a tyrosine hydroxylase promoter). In one aspect, a construct is provided which is a vector (e.g., a plasmid) useful for generating viral vectors. In one embodiment, the AAV 5′ ITR is an AAV2 ITR (i.e., AAV2-5′ITR or AAV2 5′ ITR) and the AAV 3′ITR is an AAV2 ITR (i.e., AA2V2-3′ITR or AAV2 3′ ITR). In one embodiment, the rAAV comprises an AAV capsid as described herein. In some embodiments, the rAAV comprises an AAV9.PHP.eB (AAV.PHP.eB) capsid. SEQ ID NO: 6 provides the encoded amino acid sequence of the AAV9.PHP.eB vp1 protein. In one embodiment, the rAAV comprises an AAVhu68 capsid. SEQ ID NO: 8 provides the encoded amino acid sequence of the AAVhu68 vp1 protein. Examples of nucleic acids which may be used to generate such AAVhu68 capsids and in production of rAAV viral particles comprising a vector genome are reproduced in SEQ ID NO: 7 and SEQ ID NO: 9. See, e.g., U.S. Provisional Patent Application No. 63/093,275, filed Oct. 18, 2020, International Patent Application No. PCT/US2021/055436, filed Oct. 18, 2021, which is now published as WO 2022/082109 (published Apr. 21, 2022), which are incorporated herein by reference. In other embodiments, the rAAV comprises an AAVrh91 capsid. See, e.g., WO 2020/223231, published Nov. 5, 2020, U.S. Provisional Patent Application No. 63/065,616, filed Aug. 14, 2020, U.S. Provisional Patent Application No. 63/109,734, filed Nov. 4, 2020, and International Patent Application No. PCT/US21/45945, filed Aug. 13, 2021 which is now published as WO 2022/036220A1 (published Feb. 17, 2022), which are incorporated herein by reference in their entireties.

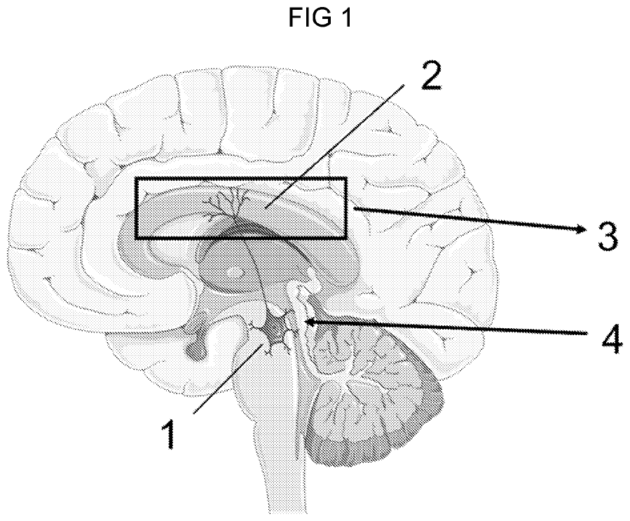

In certain embodiments, the rAAV.HPRT comprises a capsid which targets the central nervous system (CNS) and/or CNS cells. In certain embodiments, the CNS cells targeted are dopaminergic neurons. This may be done via direct delivery to the substantia nigra (SN) and/or ventral tegmental area (VTA). See, e.g., FIG. 1. Alternatively, other routes of delivery may be utilized. In certain embodiments, the rAAV comprises the vector genome which comprises a tyrosine hydroxy lase promoter for use in targeting dopaminergic neurons.

In certain embodiments, the AAV capsid is a natural Clade F AAV, e.g., AAV9, hu31, hu32, or AAVhu68. Alternatively, engineered or mutant Clade F capsids may be selected. Methods of generating vectors having the AAV9 capsid or AAVhu68 capsid, and/or chimeric capsids derived from AAV9 have been described. See, e.g., U.S. Pat. No. 7,906,111, which is incorporated by reference herein. See also, WO 2022/082109, published Apr. 21, 2022, which is incorporated herein by reference. In one embodiment, the rAAV comprises an AAVhu68 capsid. SEQ ID NO: 8 provides the encoded amino acid sequence of the AAVhu68 vp1 protein. In other embodiments, the rAAV comprises an AAV.PHP.eB capsid. SEQ ID NO: 6 provides the encoded amino acid sequence of the AAV.PHP.eB vp1 protein.

In other embodiments, the AAV capsid may selected from another clade, e.g., Clade A. In certain embodiments, the AAV capsid is selected from capsids which target the CNS (e.g., Clade F AAV (e.g., AAVhu68 or AAV9), Clade E (e.g., AAV8), or certain Clade A AAV (e.g., AAV1, AAVrh91)) capsids. See, e.g., WO 2020/223231, published Nov. 5, 2020 (rh91, including table with deamidation pattern), U.S. Provisional Patent Application No. 63/065,616, filed Aug. 14, 2020 and U.S. Provisional Patent Application No. 63/109,734, filed Nov. 4, 2020, and International Patent Application No. PCT/US21/45945, filed Aug. 13, 2021 which is now published as WO 2022/036220A 1 (published Feb. 17, 2022). In certain embodiments, the AAV capsid having reduced capsid deamidation may be selected. See, e.g., PCT/US19/19804 (which is now published as WO 2019/168961A1, published Sep. 6, 2019) filed Feb. 27, 2019 and PCT/US18/19861 (which is now published as WO 2018/160533 A1, published Sep. 7, 2018) filed Feb. 27, 2018 and incorporated by reference in their entireties. In certain embodiments, AAV capsid is selected from Clade F, E or A as a parental capsid, wherein the selected parental capsid is further modified to include the targeting peptide inserted into a hypervariable region loop of as described in U.S. Provisional Patent Application No. 63/178,881, filed Apr. 23, 2021, and International Patent Application No. PCT/US22/25879, filed Apr. 22, 2022 which are incorporated herein by reference in their entireties.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry typically in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1: US Published Patent Application No. 2009-0197338-A1: EP 1310571. See also, PCT/US19/19861, filed Feb. 27, 2019, and PCT/US19/19804, filed Feb. 27, 2019. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh. 10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector: it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV6.2, AAV7, AAV8, AAV9, AAV8 bp, AAVrh 10, AAVhu37, AAV7M8 and AAVAnc80, AAVrh90 (PCTUS20/30273, filed Apr. 28, 2020), AAVrh91 (PCTUS20/30266, filed Apr. 28, 2020), and AAVrh92, rh93, and rh91.93 (PCTUS20/30281, filed Apr. 28, 2020). See, e.g., WO 2005/033321, which is incorporated herein by reference. In one embodiment, the AAV capsid is an AAV9 variant. See, U.S. Provisional Patent Application No. 63/119,863, filed Dec. 1, 2020, and International Patent Application No. PCT/US21/61312, filed Dec. 1, 2021: US Provisional Patent Application No. 63/178,881, filed Apr. 23, 2021, and International Patent Application No. PCT/US22/25879, filed Apr. 22, 2022: U.S. Provisional Patent Application No. 63/107,030, filed Oct. 29, 2020, U.S. Provisional Patent Application No. 63/214,530, filed Jun. 24, 2021, and International Patent Application No. PCT/US2021/057201, filed Oct. 29, 2021, which is now published as WO 2022/094180 (published May 5, 2022), all of which are incorporated herein by reference. In certain embodiments, the capsid protein is designated by a number or a combination of numbers and letters following the term “AAV” in the name of the rAAV vector.

An AAVhu68 capsid is described in WO 2018/160582, which incorporated by reference in its entirety herein, and in this detailed description. In certain embodiments, an AAVhu68 capsid is further characterized by one or more of the following: AAVhu68 vp1 proteins produced by expression from a nucleic acid sequence which encodes the predicted amino acid sequence of 1 to 736 of SEQ ID NO: 8, vp1 proteins produced from SEQ ID NO: 7, or vp1 proteins produced from a nucleic acid sequence at least 70% identical to SEQ ID NO: 7 which encodes the predicted amino acid sequence of 1 to 736 of SEQ ID NO: 8; AAVhu68 vp2 proteins produced by expression from a nucleic acid sequence which encodes the predicted amino acid sequence of at least about amino acids 138 to 736 of SEQ ID NO: 8 (or SEQ ID NO: 17), vp2 proteins produced from a sequence comprising at least nucleotides 412 to 2211 of SEQ ID NO: 7 (or SEQ ID NO: 21), or vp2 proteins produced from a nucleic acid sequence at least 70% identical to at least nucleotides 412 to 2211 of SEQ ID NO: 7 (or SEQ ID NO: 21) which encodes the predicted amino acid sequence of at least about amino acids 138 to 736 of SEQ ID NO: 8 (or SEQ ID NO: 17); and/or AAVhu68 vp3 proteins produced by expression from a nucleic acid sequence which encodes the predicted amino acid sequence of at least about amino acids 203 to 736 of SEQ ID NO: 8 (or SEQ ID NO: 18), vp3 proteins produced from a sequence comprising at least nucleotides 607 to 2211 of SEQ ID NO: 7 (or SEQ ID NO: 22), or vp3 proteins produced from a nucleic acid sequence at least 70% identical to at least nucleotides 607 to 2211 of SEQ ID NO: 7 (or SEQ ID NO: 22) which encodes the predicted amino acid sequence of at least about amino acids 203 to 736 of SEQ ID NO: 8 (or SEQ ID NO: 18).

The AAVhu68 vp1, vp2 and vp3 proteins are typically expressed as alternative splice variants encoded by the same nucleic acid sequence which encodes the full-length vp1 amino acid sequence (amino acid (aa) 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence (about aa 203 to 736) without the vp1-unique region (about aa 1 to about aa 137) and/or vp2-unique regions (about aa 1 to about aa 202), or a strand complementary thereto, the corresponding mRNA or tRNA (for example, the mRNA transcribed from about nucleotide (nt) 607 to about nt 2211 of SEQ ID NO: 7 (or SEQ ID NO: 22)), or a sequence at least 70% to at least 99% (e.g., at least 85%, at least 90%, at least 95%, at least 97%, at least 98% or at least 99%) identical to SEQ ID NO: 7 which encodes aa 203 to 736 of SEQ ID NO: 8 (or SEQ ID NO: 18). Additionally, or alternatively, the vp1-encoding and/or the vp2-encoding sequence may be co-expressed with the nucleic acid sequence which encodes the AAVhu68 vp2 amino acid sequence of SEQ ID NO: 8 (about aa 138 to 736 (or SEQ ID NO: 17)) without the vp1-unique region (about aa 1 to about 137 of SEQ ID NO: 8 (or SEQ ID NO: 19)), or a strand complementary thereto, the corresponding mRNA or tRNA (for example, the mRNA transcribed from nt 412 to 2211 of SEQ ID NO: 7 (or SEQ ID NO: 21)), or a sequence at least 70% to at least 99% (e.g., at least 85%, at least 90%, at least 95%, at least 97%, at least 98% or at least 99%) identical to SEQ ID NO: 7 which encodes about aa 138 to 736 of SEQ ID NO: 8 (or SEQ ID NO: 17).

In one embodiment, the AAVhu68 vp1 nucleic acid sequence has the sequence of SEQ ID NO: 7, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 8 (about aa 203 to 736 (or SEQ ID NO: 18)) without the vp1-unique region (about aa 1 to about aa 137 of SEQ ID NO: 8 (or SEQ ID NO: 19)) and/or vp2-unique regions (about aa 1 to about aa 202 of SEQ ID NO: 8 (or SEQ ID NO: 20), or a strand complementary thereto, the corresponding mRNA or tRNA (about nt 607 to about nt 2211 of SEQ ID NO: 7 (or SEQ ID NO: 22)). In certain embodiments, also provided is a nucleic acid sequence which encodes the AAVhu68 vp2 amino acid sequence of SEQ ID NO: 8 (about aa 138 to 736 of SEQ ID NO: 8 (or SEQ ID NO: 17)) without the vp1-unique region (about aa 1 to about 137 of SEQ ID NO: 8 (or SEQ ID NO: 19)), or a strand complementary thereto, the corresponding mRNA or tRNA (nt 412 to 2211 of SEQ ID NO: 7 (or SEQ ID NO: 21)).

However, other nucleic acid sequences which encode the amino acid sequence of SEQ ID NO: 8 may be selected for use in producing rAAVhu68 capsids. In certain embodiments, the nucleic acid sequence has the nucleic acid sequence of SEQ ID NO: 7 or a sequence at least 70% to 99% identical, at least 75%, at least 80%, at least 85%, at least 90%, at least 95%, at least 97%, at least 99%, identical to SEQ ID NO: 7 which encodes SEQ ID NO: 8. In certain embodiments, the nucleic acid sequence has the nucleic acid sequence of SEQ ID NO: 7 or a sequence at least 70% to 99%, at least 75%, at least 80%, at least 85%, at least 90%, at least 95%, at least 97%, at least 99%, identical to about nt 412 to about nt 2211 of SEQ ID NO: 7 (or SEQ ID NO: 21) which encodes the vp2 capsid protein (about aa 138 to 736) of SEQ ID NO: 8 (or SEQ ID NO: 17). In certain embodiments, the nucleic acid sequence has the nucleic acid sequence of about nt 607 to about nt 2211 of SEQ ID NO: 7 (or SEQ ID NO: 22) or a sequence at least 70% to 99%, at least 75%, at least 80%, at least 85%, at least 90%, at least 95%, at least 97%, at least 99%, identical to nt 607 to about nt 2211 of SEQ ID NO: 7 (or SEQ ID NO: 22) which encodes the vp3 capsid protein (about aa 203 to 736) of SEQ ID NO: 8 (or SEQ ID NO: 18).

As used herein, the terms “rAAV” and “recombinant AAV vector” are used interchangeably, mean, without limitation, an AAV comprising a capsid protein and a vector genome packaged therein, wherein the vector genome comprising a nucleic acid heterologous to the AAV. rAAV includes “pseudotyped rAAV”, wherein the viral vector contains a vector genome containing the inverted terminal repeat of one AAV (e.g., AAV2) packaged into the capsid of a different AAV capsid protein. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 8 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine—glycine (N-G) pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins: vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine—glycine pairs.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 Jun: 78(12): 6381-6388, which identifies Clades A, B, C, D, E and F, and provides nucleic acid sequences of novel AAV, GenBank Accession Numbers AY530553 to AY530629. See, also, WO 2005/033321.

rAAV production

The rAAV described herein may be generated using techniques which are known. See, e.g., WO 2003/042397: WO 2005/033321, WO 2006/110689; U.S. Pat. No. 7,588,772 B2. Such a method involves culturing a host cell which contains a nucleic acid sequence encoding an AAV capsid: a functional rep gene: an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid: a functional rep gene: a vector genome as described; and sufficient helper functions to permit packaging of the vector genome into the AAV capsid protein. In one embodiment, the host cell is a cell culture. In another embodiment, the host cell is a suspension. In some embodiments, the host cell is a HEK 293 cell. These methods are described in more detail in WO2017/160360 A2, which is incorporated by reference herein. See also, 5,139,941: 5,741,683: 6,057,152: 6,204,059; 6,268,213: 6,491,907: 6,660,514: 6,951,753: 7,094,604: 7,172,893: 7,201,898: 7,229,823; and 7,439,065.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr 15: 20(R1): R2-R6. Published online 2011 Apr 29. doi: 10.1093/hmg/ddr141: Aucoin MG et al., Production of adeno-associated viral vectors in insect cells using triple infection: optimization of baculovirus concentration ratios. Biotechnol Bioeng. 2006 Dec 20:95(6): 1081-92; SAMI S. THAKUR, Production of Recombinant Adeno-associated viral vectors in yeast. Thesis presented to the Graduate School of the University of Florida, 2012: Kondratov O et al. Direct Head-to-Head Evaluation of Recombinant Adeno-associated Viral Vectors Manufactured in Human versus Insect Cells, Mol Ther. 2017 Aug 10. pii: S1525-0016(17)30362-3. doi: 10.1016/j.ymthe.2017.08.003. [Epub ahead of print]: Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 Feb:28(1):15-22. doi: 10.1089/hgtb.2016.164.; Li L et al. Production and characterization of novel recombinant adeno-associated virus replicative-form genomes: a eukaryotic source of DNA for gene transfer. PLOS One. 2013 Aug 1:8(8): e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 Jul: 107 Suppl:S80-93. doi: 10.1016/j.jip.2011.05.008; and Kotin RM, Large-scale recombinant adeno-associated virus production. Hum Mol Genet. 2011 Apr 15:20(R1):R2-6, doi: 10.1093/hmg/ddr141. Epub 2011 Apr 29.

A two-step affinity chromatography purification at high salt concentration followed by anion exchange resin chromatography is used to purify the rAAV product and to remove empty capsids. These methods are described in more detail in WO 2017/160360 entitled “Scalable Purification Method for AAV9”, and WO 2017/100674 entitled “Scalable Purification Method for AAV1”, which are incorporated by reference herein. In brief, the method for separating rAAV particles having packaged genomic sequences from genome-deficient AAV intermediates involves subjecting a suspension comprising recombinant AAV9 or AAV viral particles and AAV capsid intermediates to fast performance liquid chromatography, wherein the AAV9 viral particles and AAV intermediates are bound to a strong anion exchange resin equilibrated at a pH of about 10.2 for rAAV9 or about 9.8 for AAV1, and subjected to a salt gradient while monitoring eluate for ultraviolet absorbance at about 260 and about 280. In this method, the AAV full capsids are collected from a fraction which is eluted when the ratio of A260/A280 reaches an inflection point. In one example, for the affinity chromatography step, the diafiltered product may be applied to an AAV-specific resin that efficiently captures the selected AAV serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where #of GC=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330: Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus, ct al., J. Viral. (2000) 74:9281-9293). A secondary antibody is then used, one that binds to the primary antibody and contains a means for detecting binding with the primary antibody. more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., Dithiothreitol (DTT)), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or Coomassie stains. In one embodiment, the concentration of AAV vector genomes (vg) in column fractions can be measured by quantitative real time PCR (Q-PCR, q-PCR or qPCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR (oqPCR) genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther. Methods. 2014 Apr:25(2): 115-25. doi: 10.1089/hgtb.2013.131. Epub 2014 Feb 14.

Methods for determining the ratio among vp1, vp2, and vp3 of capsid protein are also available. See, e.g., Vamseedhar Rayaprolu et al., Comparative Analysis of Adeno-Associated Virus Capsid Stability and Dynamics, J Virol. 2013 Dec: 87(24): 13150-13160; Buller RM, Rose JA. 1978. Characterization of adenovirus-associated virus-induced polypeptides in KB cells. J. Virol. 25:331-338; and Rose JA, Maizel JV, Inman JK, Shatkin AJ. 1971. Structural proteins of adenovirus-associated viruses. J. Virol. 8:766-770. For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In one preferred embodiment, the rAAV comprises an AAV hu68 capsid with a vector genome comprising an AAV 5′ ITR, a CB7 promoter, a chicken beta-actin intron, a HPRT coding sequences, a rabbit beta globin polyA, spacer sequences, and an AAV 3′ ITR. In certain embodiments, a plasmid has a vector genome comprising ITRs with a shortened 130 nucleotide sequence, optionally derived from AAV2, which reverts to the full-length 145 nucleotide ITR when it replicates and is packaged into the AAV capsid. In certain embodiments, the vector genome comprises stuffer sequences between the AAV 5′ ITR and the promoter and/or the polyA and the AAV 3′ ITR. In certain embodiments, the vector genome comprises an AAV 5′ ITR, spacer sequences, sequence of nucleotide 198 to nucleotide 2788 of SEQ ID NO: 1 (or SEQ ID NO: 14), spacer sequences, and an AAV 3′ ITR. In certain embodiments, the vector genome is the full-length of SEQ ID NO: 1 (nucleotide 1 to nucleotide 3006).

As described herein, a rAAVhu68 has a rAAVhu68 capsid produced in a production system expressing capsids from an AAVhu68 nucleic acid sequence which encodes the vp1 amino acid sequence of SEQ ID NO: 8, and optionally additional nucleic acid sequences, e.g., encoding a vp3 protein free of the vp1 and/or vp2-unique regions. The rAAVhu68 resulting from production using a single nucleic acid sequence vp1 produces the heterogeneous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 8. These subpopulations include, at a minimum, deamidated asparagine (N or Asn) residues. For example, asparagines in asparagine—glycine pairs are highly deamidated. In certain embodiments, the rAAVhu68 has a rAAVhu68 capsid produced in a production system expressing capsids from an AAVhu68 nucleic acid sequence of SEQ ID NO: 7. In certain embodiments, the rAAVhu68 has a rAAVhu68 capsid produced in a production system expressing capsids from an AAVhu68 nucleic acid sequence of SEQ ID NO: 9.

It should be understood that the compositions in the vectors described herein are intended to be applied to other compositions and methods described across the specification.

### Pharmaceutical Compositions, Uses and Methods of Treatment

As described herein, Lesch-Nyhan syndrome is associated with hyperuricemia, which is typically well controlled with allopurinol, fluid, bicarbonate, hypotonia, and generalized dystonia, poor muscle control (repetitive movements: chorea), severe dysarthria, spasticity, self-injurious behavior (lip and finger biting), opisthotonos and mild intellectual disability. In some cases, behaviors include impulsive acts of aggression, spitting, or use of foul language. Some patients may also exhibit asymptomatic macrocytic anemia. Provided herein are compositions containing a rAAV and an optional carrier, excipient and/or preservative.

In certain embodiments, a composition is provided which comprises a pharmaceutically acceptable aqueous liquid and a population or stock of rAAV having a transgene useful for treatment of Lesch-Nyhan syndrome or a disorder associated with a deficiency in hypoxanthine-guanine phosphoribosyltransferase (HPRT) enzyme levels. In one embodiment, the composition is formulated for CNS-directed administration to a patient diagnosed with Lesch-Nyhan disorder, or a disease associated with a deficiency in hypoxanthine-guanine phosphoribosyltransferase (HPRT) enzyme levels. In certain embodiments, the composition is formulated for direct delivery to the dopaminergic neurons of a patient in need thereof.

Additionally provided is a pharmaceutical composition comprising a pharmaceutically acceptable carrier and a rAAV comprising a vector genome comprising a HPRT coding sequence operatively linked to regulatory elements therefor as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

In one embodiment, a composition includes a final formulation suitable for delivery to a subject/patient, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

In one embodiment, the suspension further comprises a surfactant, preservative, excipients, and/or buffer dissolved in the aqueous suspending liquid. In one embodiment, the buffer is PBS. The pH may be in the range of 6.5 to 8.5, or 7 to 8.5, or 7.5 to 8. As the pH of the cerebrospinal fluid is about 7.28 to about 7.32, for intrathecal delivery, a pH within this range may be desired: whereas for intravenous delivery, a pH of 6.8 to about 7.2 may be desired. However, other pHs within the broadest ranges and these subranges may be selected for other routes of delivery. Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. A suitable surfactant, or combination of surfactants, may be selected from among non-ionic surfactants that are nontoxic. In one embodiment, a difunctional block copolymer surfactant terminating in primary hydroxyl groups is selected, e.g., such as PluronicR F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension.

In one embodiment, composition comprising the expression cassette, vector genome, rAAV, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof.

As used herein, the term “dosage” or “amount” can refer to the total dosage or amount delivered to the subject in the course of treatment, or the dosage or amount delivered in a single unit (or multiple unit or split dosage) administration.

The composition, the suspension or the pharmaceutical compositions described herein are designed for delivery to subjects in need thereof by any suitable route or a combination of different routes. In one embodiment, the pharmaceutical composition comprises a formulation buffer suitable for delivery via intracerebroventricular (ICV), intrathecal (IT), intracisternal, administration by direct injection into the substantia nigra and/or ventral tegmental area, or intravenous (IV) routes of administration. In certain embodiments, the rAAV or the pharmaceutical composition comprises a formulation buffer suitable for intravenous, intraparenchymal (dentate nucleus), direct injection (e.g., image guided), and/or intrathecal administration to a patient in the need thereof.

In certain embodiments, the image-guided direct injection (e.g., substantia nigra ventral tegmental area) is an MRI-guided convection-enhanced injection. In certain embodiments, a convection-enhanced delivery (CED) refers to the use of a pressure gradient to generate bulk flow within the brain parenchyma, i.e., convection of composition within the interstitial fluid driven by infusing a solution through a cannula placed directly in the targeted structure. This method allows therapeutic agents to be homogenously distributed through large volumes of brain tissue by bypassing the blood brain barrier and surpassing simple diffusion (Richardson, et al., 2011, Novel Platform for MRI-Guided Convection-Enhanced Delivery of Therapeutics: Preclinical Validation in Nonhuman Primate Brain, Stereotact. Funct. Neurosurg. 89(3):141-151, which is incorporated herein by reference in its entirety). See also, Kalkowski, L., et al., 2018, MRI-guided intracerebral convection-enhanced injection of gliotoxins to induce focal demyelination in swine, PLOS One, 13(10): e0204650: WO2016073693A2; and Prezelski, K., et al., 2021, Design and Validation of a Multi-Point Injection Technology for MR-Guided Convection Enhanced Delivery in the Brain, Front. Med. Technol., 14(3):725844, which are incorporated herein by reference in their entireties.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, intracerebroventricular (ICV or icv) suboccipital/intracisternal, and/or C1-2 puncture. For example, material may be introduced for diffusion throughout the subarachnoid space by means of lumbar puncture. In another example, injection may be into the cisterna magna (intracisternal magna: ICM). Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther. Methods Clin Dev. 2014: 1: 14051. Published online 2014 Dec 10, doi: 10.1038/mtm.2014.51. As used herein, the terms “intracisternal delivery” or “intracisternal administration” refer to a route of administration for drugs directly into the cerebrospinal fluid of the brain ventricles or within the cisterna magna cerebellomedullaris, more specifically via a suboccipital puncture or by direct injection into the cisterna magna or via permanently positioned tube.

As used herein, the term “intraparenchymal (dentate nucleus)” or IDN refers to a route of administration of a composition directly into dentate nuclei. IDN allows for targeting of dentate nuclei and/or cerebellum. In certain embodiments, the IDN administration is performed using ClearPoint® Neuro Navigation System (MRI Interventions, Inc., Memphis, TN) and ventricular cannula, which allows for MRI-guided visualization and administration. Alternatively, other devices and methods may be selected.

In certain embodiments, the rAAV or composition thereof is administered intraparenchymally with a method which comprises using the ClearPoint R injection system wherein the system consists of a monitor to visualize the brain and injection procedure in real time, a head fixation frame that is secured to the skull, and an MRI-compatible SmartFrameR (MRI Interventions Inc., Memphis, TN) trajectory device that enables MRI-guided alignment during the procedure. This system allows for the direct injection to be combined with real-time visualization of the injection tract by MRI. To enable visualization of rAAV or composition distribution, the injection material containing the vector is mixed with gadolinium, which is contrast agent (final concentration of 1-2 mM gadolinium). During the direct injection procedure, the injection cannula is placed through the ClearPoint® frame to the correct position on the skull and the frame maintains the correct trajectory. The final position of the injection cannula is confirmed using real-time MRI images, and then the rAAV or composition is injected into the parenchyma of the deep cerebellar nuclei using convection-enhanced delivery. Each subject receives administration of the rAAV or composition plus gadolinium in each dentate nucleus injected at a rate of 0.5 μL/min initially, and then at an increased rate of up to 5 μL/min based on clinician discretion during the procedure. The procedure takes approximately 5-6 hours and subjects are anesthetized for the duration of the procedure.

In some embodiments, the rAAV or composition is administered via unilateral and/or bilateral MRI guided direct injection into the deep cerebellar nuclei (DCN) via convection-enhanced delivery (CED). In certain embodiments, the rAAV or composition is delivered using ClearpointR NeuroNavigation system and Smartflow Cannulas, adapted for DCN injection.

In certain embodiments, the compositions can be formulated in dosage units to contain an amount of rAAV that is in the range of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In certain embodiments, the compositions can be formulated in dosage units to contain an amount of rAAV that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹. 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³. 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵. 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In certain embodiments, the rAAV, the composition, the suspension or the pharmaceutical composition is used in preparing a medicament. In certain embodiments, uses of the same are for treatment of Lesch-Nyhan syndrome or disorder associated with a deficiency in HPRT enzyme levels in a subject in need thereof are provided.

As used herein, the term “treatment” or “treating” is defined encompassing administering to a subject one or more compounds or compositions described herein for the purposes of amelioration of one or more symptoms of Lesch-Nyhan syndrome or disorder associated with a deficiency in HPRT enzyme levels. “Treatment” can thus include one or more of reducing onset or progression of Lesch-Nyhan disease, reducing the severity of the symptoms, removing the disease symptoms, delaying progression of disease, or increasing efficacy of therapy in a given subject.

In certain embodiments, the composition is useful for treatment conditions and/or symptoms associated with Lesch-Nyhan syndrome, or a disorder associated with deficiency in HPRT enzyme levels. In certain embodiments, the composition is useful for treatment of neurobehavioral conditions or symptoms associated with Lesch-Nyhan syndrome or a disorder associated with deficiency in HPRT enzyme levels. In some embodiments, the neurobehavioral symptoms include, but not limited to, self-injurious behavior (lip and finger biting), intellectual disability, impulsive acts of aggression, spitting, or use of foul language.

In certain embodiments, the composition is useful for treatment of musculoskeletal symptoms associated with Lesch-Nyhan syndrome or a disorder associated with deficiency in HPRT enzyme levels. In some embodiments, the musculoskeletal conditions include, but not limited to, hypotonia, generalized dystonia, poor muscle control (repetitive movements: chorea), severe dysarthria, spasticity. In certain embodiments, the composition is useful for treatment hyperuricemia and/or hyperuricemia-related symptoms associated with Lesch-Nyhan syndrome, or a disorder associated with deficiency in HPRT enzyme levels.

In certain embodiments, provided herein is a method of treating Lesch-Nyhan syndrome, or a disorder associated with deficiency in HPRT enzyme levels by administering to a subject in need thereof an rAAV that provides HPRT coding sequence and results in expression of functional HPRT in a dopaminergic neuron. In certain embodiments, provided herein is a method of treating Lesch-Nyhan syndrome, or a disorder associated with deficiency in HPRT enzyme levels by administering to a subject in need thereof an rAAV that provides HPRT coding sequence and results in expression of functional HPRT in the substantia nigra and/or ventral tegmental area. In certain embodiments, the method includes administering to a mammalian subject in need thereof, a pharmaceutically effective amount of a composition comprising a rAAV comprising an HPRT coding sequence operably linked to the control of regulatory sequences, and a pharmaceutically acceptable carrier. In one embodiment, such a method is designed for treating, retarding or halting progression of Lesch-Nyhan syndrome in a mammalian subject.

In one embodiment, a rAAV is delivered about 1×10¹⁰ to about 1×10¹⁵ genome copies (GC)/kg body weight. In certain embodiments, the subject is human. In one embodiment, the rAAV is administered more than one time. In a further embodiment, the rAAV is administered days, weeks, months or years apart.

Any suitable route of administration may be selected, for rAAV.HPRT, e.g., intravenous, intrathecal, intracerebroventricular, or direct injection. Conventional and pharmaceutically acceptable routes of administration include, but are not limited to, direct delivery to a desired organ (e.g., brain, CSF, substantia nigra (SN), ventricular tegmental area (VTA)), oral, inhalation, intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired.

In one embodiment, a method for treating Lesch-Nyhan syndrome involves direct delivery of an active drug for Lesch-Nyhan syndrome to the dopaminergic neurons of a patient. In certain embodiments, the active drug is a therapeutic gene, optionally delivered via a vector. In certain embodiments, direct delivery (e.g., intraparenchymal) involves injection into the substantia nigra and/or ventral tegmental area. Suitably, the injection is guided via imaging (e.g., magnetic resonance imaging (MRI)). In certain embodiments, a method for treating Lesch-Nyhan syndrome involves delivery of an active drug or a pharmaceutical composition described herein using Ommaya reservoir device.

In certain embodiment, the active drug is a therapeutic gene. In certain embodiments, the gene is hypoxanthine-guanine phosphoribosyltransferase (HPRT). In certain embodiment, the gene is delivered via a vector. In certain embodiments, the gene is the rAAV.HPRT as described herein. In certain embodiments, the gene therapy vector provided herein may be combined in a therapy with another active drug, e.g., a small molecule and is selected from allopurinol, ecopipam, carbidopa/levodopa, diazepam, phenobarbital, or haloperidol, and benzodiazepines or baclofen (ninds.nih.gov/Disorders/All-Disorders/Lesch-Nyhan-Syndrome-Information-Page: Jinnah H. A., Lesch-Nyhan disease: from mechanism to model and back again, Disease Models & Mechanisms, 2009, 2:116-121: Khasnavis, T., et al., A double-blind, placebo-controlled, crossover trial of the selective dopamine DI receptor antagonist ecopipam in patients with Lesch-Nyhan disease, Mol Genet Metab, 2016, 118(3): 160-166).

In certain embodiments, a method for treating Lesch-Nyhan syndrome involves co-administration of two or more of therapeutic drugs, e.g., therapeutic gene and small molecule. In certain embodiments, method for treating Lesch-Nyhan syndrome involves co-administration of therapeutic gene, e.g., rAAV.HPRT, and allopurinol.

A patient's status may be monitored by assessing brain morphology, dopamine expression levels, and/or dopaminergic cell bodies. More particularly, Lesch-Nyhan patients have primarily morphologically normal brains, but have decreased dopamine in the striatum. This may be assessed using PET studies. Additionally, patients exhibiting disease symptoms have abnormal dopamine metabolism as reflected in central spinal fluid (CSF) metabolites and/or the presence of dopaminergic cell bodies and/or decreased dopamine release. Further, abnormalities of nigrostriatal dopamine pathway appear to correlate with the motor phenotype of the disease.

Thus, in certain embodiments, treatment of a Lesch-Nyhan patient may involve assessing brain morphology via a suitable imaging method (e.g., positron emission tomography (PET) study), coupled with assessment of dopamine levels in CSF, another suitable bodily fluid, or using imaging and a dopamine tracer (e.g., LBT-999). A variety of dopamine tracers are known and may be selected, e.g., cocaine derivative (E)-N-(4-fluorobut-2-enyl)2β-carbomethoxy-3β-(4′-tolyl)nortropane, or LBT-999, [18F]LBT-999, or its non-fluorinated derivative (E)-N-(3-iodoprop-2-enyl)-2-carbomethoxy-3-(4-methylphenyl) nortropane, or PE2I. Optionally, other imaging systems may be selected and used. In certain embodiments, a comparative study of brain morphology and dopamine metabolism is performed in a patient, prior to treating, at the time of initiating treatment, and/or at a selected time period post-initiation of treatment. This method may be performed with the rAAV.HPRT vectors provided herein. Additionally or alternatively, this method may be performed with other therapeutics for treating Lesch-Nyhan disease. Such other therapeutics may be other AAV-mediated treatments, other vector-mediated treatments, or non-vector mediated treatments.

As used herein, a “therapeutically effective amount” refers to the amount of a composition (e.g., rAAV.hHPRT composition) that delivers and expresses in the target cells an amount of enzyme sufficient to reach therapeutic goal. In certain embodiments, the therapeutic goal is to ameliorate or treat one or more of the symptoms of Lesch Nyhan disease and/or disorder associated with a deficiency in hypoxanthine-guanine phosphoribosyltransferase (HPRT) enzyme levels. In certain embodiments, the therapeutic goal for treating Lesch Nyhan syndrome and/or disorder associated with a deficiency in hypoxanthine-guanine phosphoribosyltransferase (HPRT) enzyme levels is to restore HPRT expression in a dopaminergic neuron, to the functional level in a patient that is in the normal range or to the non-Lesch Nyhan syndrome level. In another embodiment, therapeutic goal is to increase the HPRT to at least about 99%, about 95%, about 90%, about 85%, about 80%, about 75%, about 70%, about 65%, about 60%, about 55%, about 50%, about 45%, about 40%, about 45%, about 40%, about 35%, about 30% about 25%, about 20%, about 15%, about 10%, about 5%, about 2%, about 1% of the normal or non-Lesch-Nyhan syndrome level, or as compared to levels of HPRT expression before treatment. Patients rescued by delivering HPRT function to less than 100% activity levels may optionally be subject to further treatment (e.g., co-administering small molecule active drug). In another embodiment, therapeutic goals are to increase the HPRT expression in a percentage of target dopaminergic neurons, including about 60%, about 55%, about 50%, about 45%, about 40%, about 45%, about 40%, about 35%, about 30% about 25%, about 20%, about 15%, about 10%, about 5%, about 2%, or about 1% of dopaminergic neurons in a selected population. An effective amount may be determined based on an animal model, rather than a human patient.

As used herein, a recombinant viral vector is any suitable viral vector which targets the desired cell(s). Thus, the recombinant viral vectors described herein preferably target one or more of the cells and tissues affected by Lesch-Nyhan disease, including cells of the central nervous system (e.g., brain). The examples provide illustrative recombinant adeno-associated viruses (rAAV). However, other suitable viral vectors may include, e.g., a recombinant adenovirus, a recombinant parvovirus such a recombinant bocavirus, a hybrid AAV/bocavirus, a recombinant herpes simplex virus, a recombinant retrovirus, or a recombinant lentivirus. In preferred embodiments, these recombinant viruses are replication-defective.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV nuclease (e.g., DNase)-resistant particle having an AAV protein capsid into which is packaged expression cassette flanked by AAV inverted terminal repeat sequences (ITRs) for delivery to target cells. A nuclease-resistant recombinant AAV (rAAV) indicates that the AAV capsid has fully assembled and protects these packaged vector genome sequences from degradation (digestion) during nuclease incubation steps designed to remove contaminating nucleic acids which may be present from the production process. In many instances, the rAAV described herein is DNase resistant. A “replication-defective” virus or viral vector refers to a synthetic or artificial viral particle in which an expression cassette containing a gene of interest is packaged in a viral capsid or envelope, where any viral genomic sequences also packaged within the viral capsid or envelope are replication-deficient: i.e., they cannot generate progeny virions but retain the ability to infect target cells. In one embodiment, the genome of the viral vector does not include genes encoding the enzymes required to replicate (the genome can be engineered to be “gutless”-containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

“Plasmid” or “plasmid vector” generally is designated herein by a lower-case p preceded and/or followed by a vector name. Plasmids, other cloning and expression vectors, properties thereof, and constructing/manipulating methods thereof that can be used in accordance with the present invention are readily apparent to those of skill in the art. In one embodiment, the elements of a vector genome as described herein or the expression cassette as described herein are engineered into a suitable genetic element (a vector) useful for generating viral vectors and/or for delivery to a host cell, e.g., naked DNA, phage, transposon, cosmid, episome, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY. The term “transgene” or “gene of interest” as used interchangeably herein means an exogenous and/or engineered protein-encoding nucleic acid sequence that is under the control of a promoter and/or other regulatory elements in an expression cassette, rAAV genome (or rAAV vector genome), recombinant plasmid or production plasmid, vector, or host cell described in this specification.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in the same relationship to each other in nature.

As used herein, the term “host cell” may refer to the packaging cell line in which a vector (e.g., a recombinant AAV or rAAV) is produced from a production plasmid. In the alternative, the term “host cell” may refer to any target cell in which expression of a gene product described herein is desired. Thus, a “host cell,” refers to a prokaryotic or eukaryotic cell (e.g., human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. In a further embodiment, the term “host cell” is a dopaminergic neuron, e.g., a dopaminergic neuron of the CNS. As used herein, the term “target cell” refers to any target cell in which expression of a heterologous nucleic acid sequence or protein is desired. In certain embodiments, the target cell is a dopaminergic neuron of the CNS, in particular a dopaminergic neuron with a mutated or defective gene encoding HPRT or a dopaminergic neuron that lacks HPRT expression.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used herein, “disease”, “disorder”, and “condition” are used interchangeably, to indicate an abnormal state in a subject. In one embodiment, the disease is Lesch-Nyhan disease.

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human.

The term “expression” is used herein in its broadest meaning and comprises the production of RNA, of protein, or of both RNA and protein. With respect to RNA, the term “expression” or “translation” relates in particular to the production of peptides or proteins. Expression may be transient or may be stable.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13): 2682-2690 (1999).

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an”, refers to one or more, for example, “a dopaminergic neuron”, is understood to represent one or more dopaminergic neuron(s). As such, the terms “a” (or “an”), “one or more,” and “at least one” is used interchangeably herein.

As used herein, the term “about” means a variability of plus or minus 10% from the reference given, unless otherwise specified.

In certain instances, the term “E+#” or the term “e+#” is used to reference an exponent. For example, “5E10” or “5e10” is 5×10¹⁰. These terms may be used interchangeably.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention. Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are illustrative only and are not a limitation on the invention described herein.

Lesch-Nyhan syndrome (also referred to as Lesch-Nyhan disease, Lesch Nyhan syndrome, Lesch Nyhan disease, or LNS) is a rare, X-linked recessive neurological condition with prevalence of 1:300,000, which is caused by a loss-of-function mutation in a gene encoding hypoxanthine-guanine phosphoribosyltransferase (HPRT). HPRT enzyme is involved in purine salvage pathway. Lesch-Nyhan disease is characterized by an overproduction of uric acid, which may lead to a gouty arthritis, subcutaneous gouty tophi and nephrolithiasis, and is characterized by numerous neurological and/or behavioral abnormalities (see also, Jinnah H. A., Lesch-Nyhan disease: from mechanism to model and back again, Disease Models & Mechanisms, 2009, 2:116-121). Phenotypes in patients with Lesch-Nyhan syndrome may include but not limited to hyperuricemia, hypotonia, generalized dystonia, severe dysarthria, compulsive self-injurious behavior, and mild intellectual disability. The neurological manifestations of LNS may be related to defective projections of dopaminergic neurons of the substantia nigra, which are apparent on positron emission tomography studies of LNS patients. LNS results from mutation or deletion of the hypoxanthine-guanine phosphoribosyltransferase (HPRT) gene, leading to enzyme deficiency. HPRT is an enzyme that functions to recycle nucleotides: however, it remains unclear how disruption of this pathway leads to dysfunction of dopaminergic neurons and pathology. Currently there is no approved treatment for LNS.

An importance of function of dopamine-neuron terminals in striatum of patients with Lesch-Nyhan disease has been observed. Patients with Lesch-Nyhan syndrome have (mostly) morphologically normal brains; however, the brain tissue does have decreased dopamine levels in striatum. This was confirmed in analysis of postmortem tissue, in PET studies in living subject using dopamine tracers, and abnormal dopamine metabolism have been observed in CSF metabolites. Additionally, the observed abnormalities of nigrostriatal dopamine pathway fit with motor phenotype of Lesch-Nyhan syndrome. In the studies below, we examine effect of postnatal HPRT restoration in dopaminergic cells in aiding to restore striatal dopamine levels using a gene therapy approach of rAAV.HPRT direct delivery to dopaminergic neurons, in substantia nigra (SN) and ventral tegmental area (VTA).

### Example 1: Generation of rAAV.HPRT

A cis plasmid for use in producing an rAAV viral particle was designed to have a vector genome of shortened 130 nucleotide AAV2-5′ ITR, spacer sequences, a CB7 promoter, a chicken beta-actin intron, a HPRT coding sequences, a rabbit beta globin polyA, spacer sequences, and a shorted 130 nucleotide AAV2-3′ ITR. The cis plasmid contains plasmid elements and a vector genome having the sequence of nucleotide 1 to nucleotide 3006 of SEQ ID NO: 1 suitable for packaging into a capsid (comprising expression cassette of SEQ ID NO: 14).

rAAV having an AAV.PHP.eB capsid (amino acid sequence of SEQ ID NO: 6, encoded by the nucleic acid sequence of SEQ ID NO: 5) having this vector genome was generated for use in mouse studies in a production host cell also transfected with a plasmid which expresses an AAV.PHP.eB capsid, AAV rep sequences necessary for replication of the vector genome which is deleted of AAV cap and AAV rep coding sequences, and expressing adenovirus helper functions necessary for replication and packaging. Conventional triple transfection techniques were utilized. The resulting rAAV.PHP.eB.hHPRT vectors are utilized in mouse studies.

rAAV.hHPRT having an AAVhu68 capsid (amino acid sequence of SEQ ID NO: 8) are generated using a plasmid carrying this vector genome and similar techniques, but using a plasmid expressing an AAVhu68 capsid (SEQ ID NO: 7 or SEQ ID NO: 9) rather than AAV.PHP.eB.

### Example 2: HPRT expression and activity in vitro

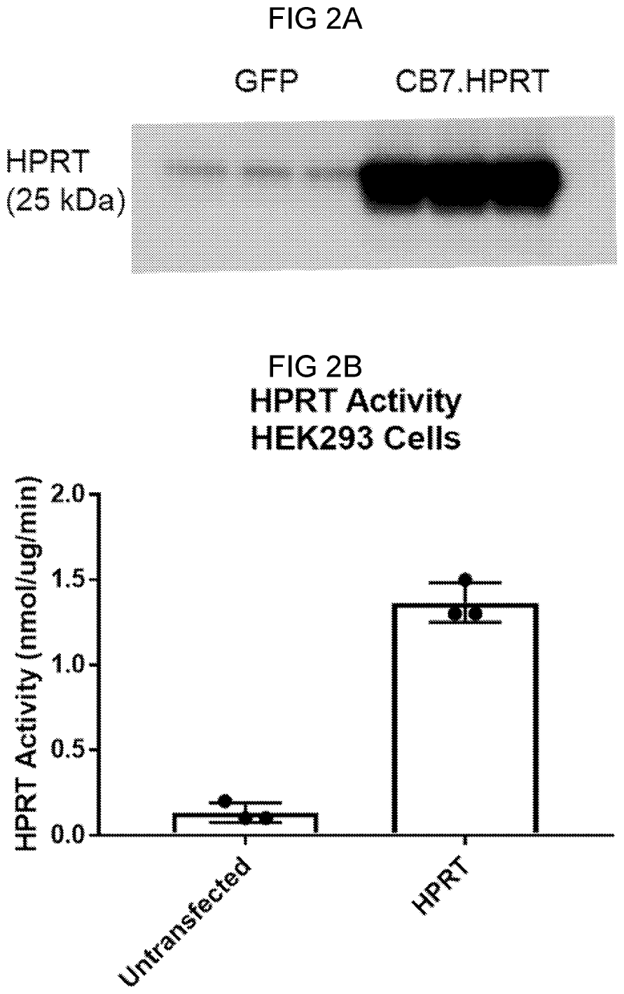

In this study, HEK293 cells were transfected with 3 μg pAAV.CB7.CI.HPRT.RBG generated as described in Example 1. Vectors were harvested from 6-well plates 24 hr after transfection. Samples processed with Sephadex desalting columns to remove background NADH. GFP comparison wells used as transfection control. HPRT activity measured from IMP concentration (pmol/well) after 30 minutes. FIGS. 2A and 2B show hypoxanthine-guanine phosphoribosyltransferase (HPR) expression and activity in vitro. FIG. 2A shows western blot results of HPRT expression levels measured in harvested samples of HEK293 cells transfected with 3 μg of pAAV.CB7.HPRT.RBG plasmid, wherein GFP-transgene (green fluorescent protein) comprising plasmid was used as a transfection control. FIG. 2B shows HPRT activity levels measured from inosine monophosphate (IMP) concentration (pmol/well) after 30 min in transfected HEK293 cells.

### Example 3: AAV-Mediated HPRT expression in brain

In this study, we evaluated AAV-mediated HPRT expression in adult mouse brain in restoration of dopamine levels in adult mouse brain following postnatal HPRT expression. We have utilized adeno-associated virus (AAV) gene replacement of HPRT as a strategy for enzyme replacement and restoration of dopamine levels in a mouse model of LNS. See, FIG. 1, which shows gene therapy strategy for Lesch-Nyhan syndrome, wherein the AAV-HPRT is directly injected into the substantia nigra (SN) targeting the cell bodies of the dopaminergic neurons. HPRT expression in the SN leads to increased dopamine release at synaptic terminals. Dopamine levels are measured within the striatum. A dose of 100 μL of vehicle or rAAV.PHP.eB.CB7.CI.hHPTR.rBG (3×10¹¹ GC) was injected intravenously into the tail vein of male HPRT knock-out (KO) mice (3 months old at injection). The HPRT KO mouse model demonstrates no neuronal phenotype, but has decreases striatal dopamine with a morphologically normal brain. If HPRT restoration corrects striatal dopamine deficient in the mice, a clinical trial can test the same outcome in humans, e.g., via PET imaging.

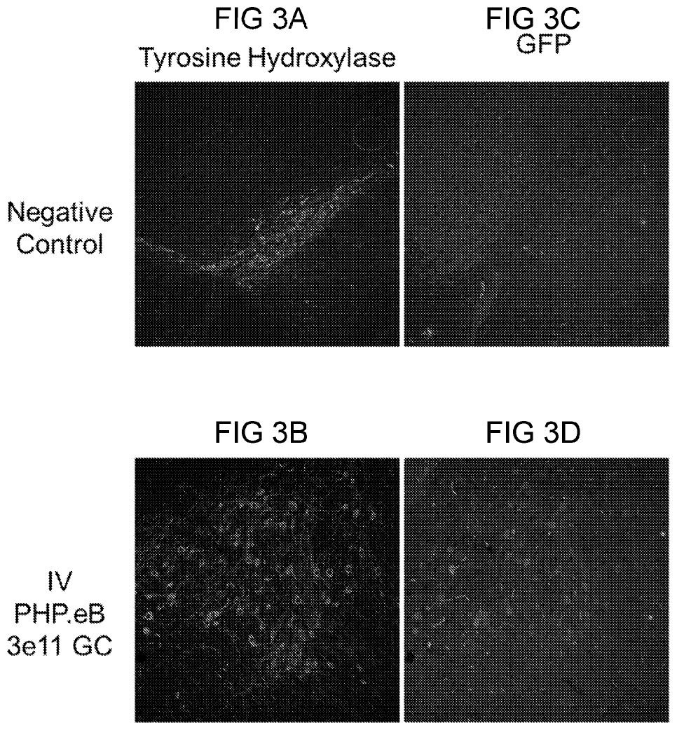

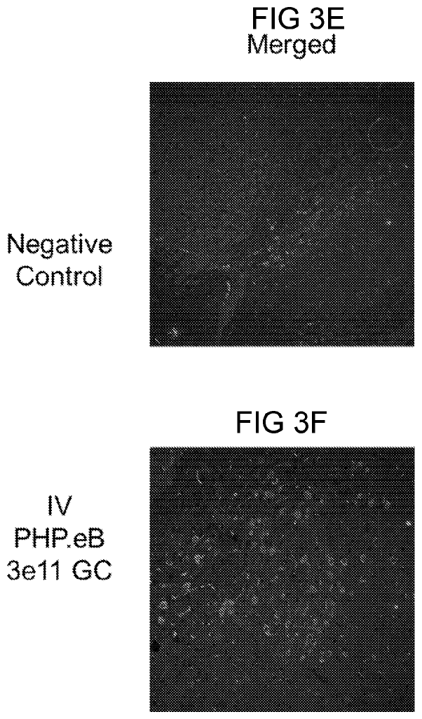

Four week-post administration of rAAV-PHP.eB.CB7.CI.hHPTR.rBG, HPRT expression levels were assessed using western blot and microscopy imaging analysis. Additionally, HPRT expression levels are assessed in dopaminergic neurons, e.g., using a marker for tyrosine hydroxylase which identifies dopaminergic neurons in CNS. See, FIG. 3, which shows expression of dopaminergic neurons in adult mice. FIGS. 3A to 3F show results of microscopy study using AAV9.PHP.eB (or negative control) in targeting dopaminergic neurons in adult mice stained with tyrosine hydroxylase (red) or green fluorescent protein (GFP). FIG. 3A shows results of microscopy study using negative control in adult mice stained with tyrosine hydroxylase (red). FIG. 3B shows results of microscopy study using AAV9.PHP.eB (IV, 3e11 (3×10¹¹ GC)) in targeting dopaminergic neurons in adult mice stained with tyrosine hydroxylase (red). FIG. 3C shows results of microscopy study using negative control in adult mice stained with green fluorescent protein (GFP). FIG. 3D shows results of microscopy study using AAV9.PHP.eB (IV, 3e11 (3×10¹¹ GC)) in targeting dopaminergic neurons in adult mice stained with green fluorescent protein (GFP). FIG. 3E shows merged results of microscopy study using or negative control in adult mice stained with tyrosine hydroxylase (red) or green fluorescent protein (GFP). FIG. 3F shows merged results of microscopy study using AAV9.PHP.eB (IV, 3e11 (3×10¹¹ GC)) in targeting dopaminergic neurons in adult mice stained with tyrosine hydroxylase (red) or green fluorescent protein (GFP).

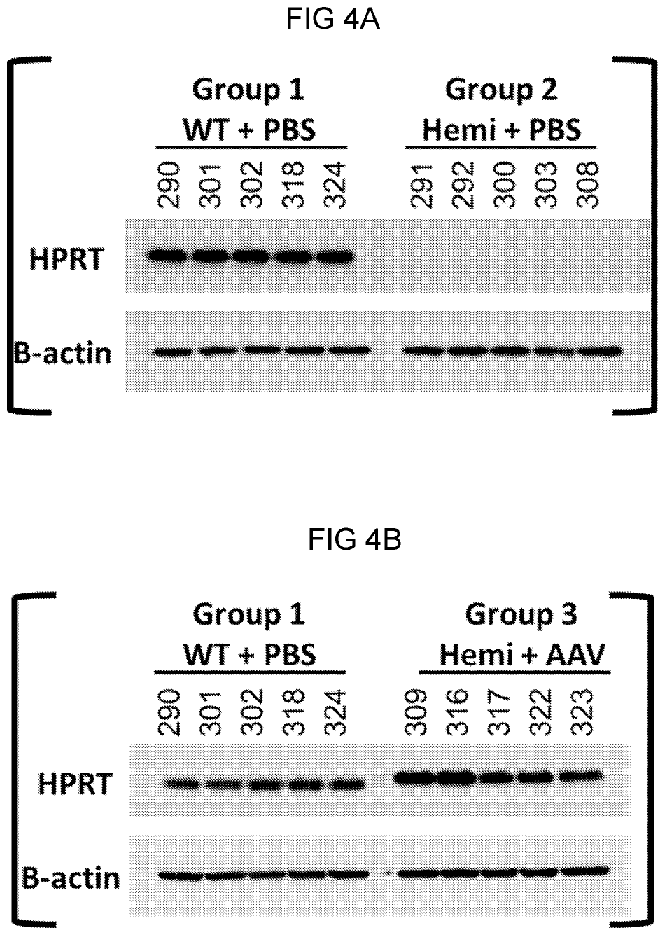

Treatment with rAAV.PHP.eB.hHPRT restored HPRT expression in the brain of the HPRT knock-out mice (KO). See, FIG. 4. FIGS. 4A and 4B show restoration of HPRT expression in adult mouse brain following intravenous administration of rAAV.PHP.eB comprising HPRT transgene. FIG. 4A shows restoration of HPRT expression in adult mouse brain following intravenous administration of AAV9.PHP.eB comprising HPRT transgene, in Groups 1 and 2. FIG. 4B shows restoration of HPRT expression in adult mouse brain following intravenous administration of AAV9.PHP.eB comprising HPRT transgene, in Groups 1 and 3. These data show that a widespread expression of HPRT in the brain of HPRT KO mice restored dopamine levels in the striatum to normal.

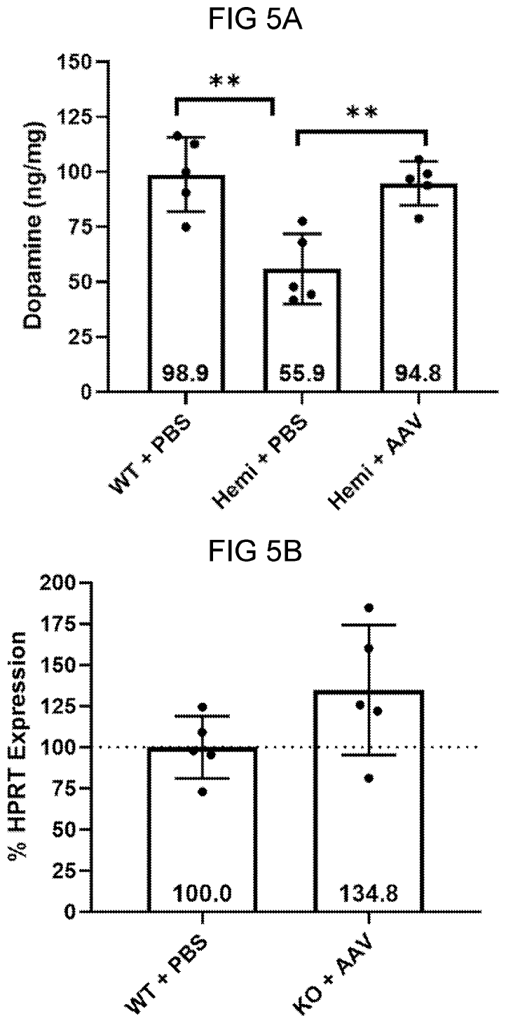

Additionally, we observed that post-natal HPRT expression reversed striatal dopamine deficit in adult HPRT knock-out mice. See, FIGS. 5A and 5B. FIGS. 5A and 5B shows delivery of AAV.PHP.eB.HPRT results in rescue of dopamine levels in the striatum of HPRT KO mice (plotted as concentration of dopamine (ng/mg): WT (wild type)+PBS (98.9), Hemi+PBS (55.9), and Hemi+AAV (94.8)). FIG. 5A shows dopamine levels in adult HPRT knock out mice (KO) treated with rAAV.PHP.eB.hHPRT, plotted as HPLC measurement of dopamine levels in the striatum of mouse brains. FIG. 5B quantification of western blot of whole brain probed with HPRT or ß-actin antibodies (plotted as percent HPRT expression: WT+PBS (100.0%), and KO+AAV (134.8%)).

Next, we evaluate transgene delivery to dopaminergic cell bodies with stereotaxic (i.e., direct delivery to the Substantia Nigra (SN)) delivery of rAAVhu68.CB7.eGFP and rAAVhu68.hHPRT, and intravenous delivery of rAAV9.PHP.eB.TH.hHPRT, wherein TH is tyrosine hydroxylase promoter. Additionally, we evaluate dopamine synthesis, transport and dynamics using positron emission tomography (PET) using PET-tracer in mice following treatment with rAAVhu68.hHPRT or rAAV9.PHP.eB.hHPRT. Furthermore, rAAVhu68.hHPRT is evaluated in non-human primates (NHP).

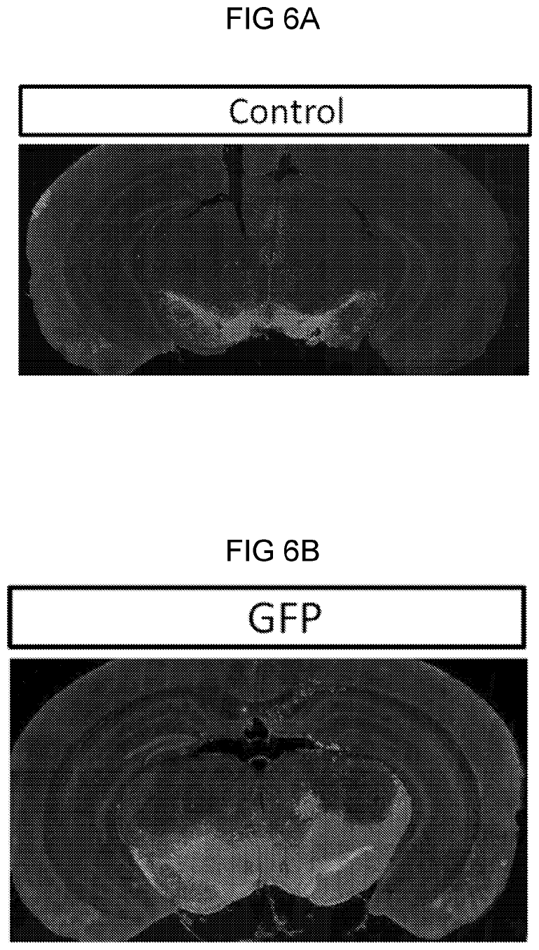

Briefly, a wildtype mice received bilateral injections into the SN using stereotaxic coordinates. Mice received 0.6 to 1.0 μL of AAVhu68.Cb7.eGFP with doses ranging from about 6×10⁸ (6e8) to 1×10⁹ (1e9) GC per side. FIGS. 6A and 6B shows delivery of AAV-GFP to the SN in mice. FIG. 6A shows coronal sections of mouse brain from control mouse brain stained with tyrosine hydroxylase (TH, red) to highlight the SN. FIG. 6B shows coronal section of mouse brain from AAV-GFP injected mouse brain (GFP, green).

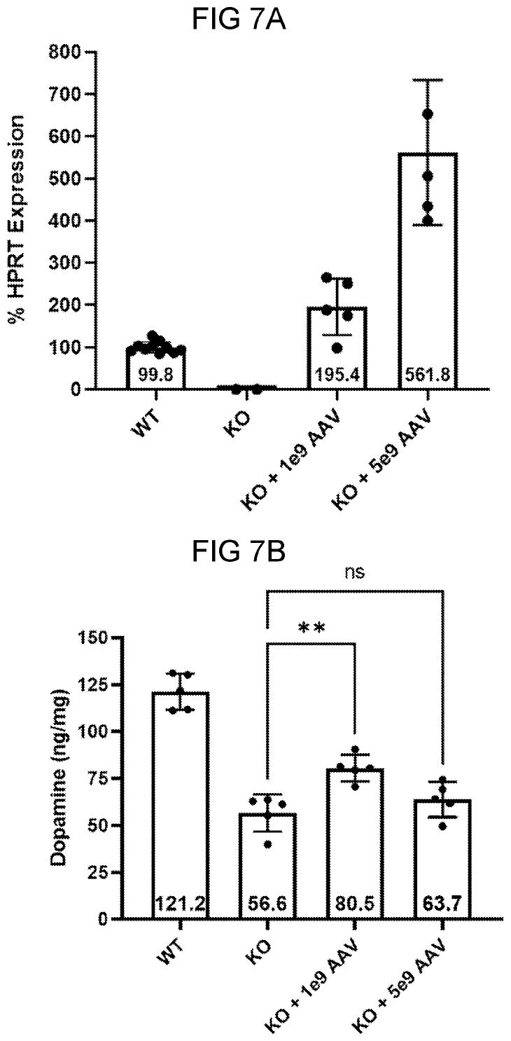

Furthermore, we examined HPRT expression and dopamine levels following a stereotaxic intraparenchymal injection of AAV.HPRT in KO Mice. Briefly, a 3-month-old HPRT KO mice received an AAVhu68.Cb7.HPRT via stereotaxic intraparenchymal injection to the SN. We observed that a restoration of HPRT expression in SN improved dopamine levels in the striatum. FIGS. 7A and 7B shows intraparenchymal delivery of AAV-HPRT to SN in HPRT KO mice. FIG. 7A shows quantitation of Western blot measuring HPRT expression in the SN (plotted as percent HPRT expression: WT (99.8%), KO (N/A), KO+1e9AAV (195.4%), and KO+5e9AAV (561.8%)). FIG. 7B shows HPLC measurement of dopamine levels in the striatum (plotted as concentration of dopamine (ng/mg): WT (121.2), KO (56.6), KO+1e9AAV (80.5), KO+5e9AAV (63.7)).

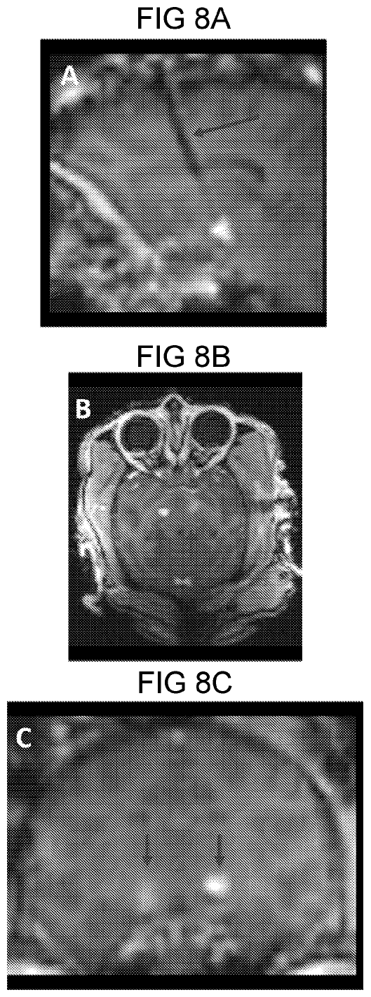

Additionally, we performed an MRI-guided AAV delivery to the SN in a nonhuman primate (NHP). Briefly, a nonhuman primate was injected bilaterally at coordinates for the SN. Injections were well tolerated and had no adverse effects. FIGS. 8A to 8C shows MRI-guided intraparenchymal delivery of rAAV.HPRT to SN in a nonhuman primate. FIG. 8A shows sagittal view showing needle track (arrow) and infusion site. FIG. 8B shows axial view showing left and right infusion sites (arrows). FIG. 8C shows coronal view showing two infusion sites (arrows).

These data show showed that injection of an rAAV.HPRT transgene into the SN of HPRT KO mice restored enzyme function and dopamine levels in the brain. Further, NHP studies assess vector toxicity and expression following intraparenchymal delivery to the brain. Overall, this gene replacement strategy shows correction of the dopaminergic defect is possible and could alleviate symptoms in LNS patients.

MRI-guided AAV delivery—Device Description

For the MRI-guided direct injection rAAV.hHPRT into the parenchyma (i.e., SN and or VTA) is performed via convection-enhanced delivery (CED). MRI-CED administration utilizes the Clearpoint® NeuroNavigation system and Smartflow Cannualas. All syringes, syringe pumps, and associated devices are CE marked, and a declaration of conformity and/or EC certificate are provided in the IMPD. The direct injection procedure using the ClearPoint injection system is performed by a neurosurgeon who has been trained and found to be proficient in the use of the ClearPoint system. Training is provided by ClearPoint Neuro, Inc., and a preceptor can be provided by ClearPoint Neuro, Inc. for initial injections if needed. The ClearPoint injection system consists of a monitor to visualize the brain and injection procedure in real time, a head fixation frame that is secured to the skull, and an MRI-compatible SmartFrame trajectory device that enables MRI-guided alignment during the procedure. This system allows for the direct injection to be combined with real-time visualization of the injection tract by MRI. To enable visualization of vector distribution, the injection material containing the vector is mixed with gadolinium (final concentration of 2 mM gadolinium). Proper precautions are taken with the gadolinium, including warning subjects of the potential risks of gadolinium use and prolonged gadolinium retention for brain MRI in informed consent forms. During the direct injection procedure, the injection cannula is placed through the ClearPoint frame to the correct position on the skull, and the frame is maintained the correct trajectory. The final position of the injection cannula is confirmed using real-time MRI images, and then the vector is injected into the parenchyma (substantia nigra (SN) and/or ventral tegmental area (VTA)) using convection-enhanced delivery. Each subject receives administration of the rAAV.HPRT plus gadolinium in SN injected at a rate of 0.5 μL/minute initially, and then at an increased rate of up to 5 μL/minute based on clinician discretion during the procedure. It is expected that the procedure takes approximately 5-6 hours and that subjects are anesthetized for the duration of the procedure.

### Table (Sequence Listing Free Text)

The following information is provided for sequences containing free text under numeric identifier <223>.

| SEQ ID NO: | |
|---|---|
| (containing free text) | Free text under <223> |
| 1 | <223> vector genome CB7.CI.HPRT.RBG |
| 2 | <223> Synthetic Construct |
| 3 | <223> engineered nucleic acid sequence |
| | HPRT |
| 4 | <223> amino acid sequence HPRT |
| 10 | <223> CB7 hybrid promoter |
| 11 | <223> chicken beta actin intron |
| 12 | |
| 13 | <223> WPRE element (mut) |
| 14 | <223> expression cassette |
| | CB7.CI.HPRT.RBG |
| | <220> |
| | <221> enhancer |
| | <222> (1) . . . (382) |
| | <223> CMV IE enhancer |
| | <220> |
| | <221> promoter |
| | <222> (1) . . . (666) |
| | <223> CB7 hybrid promoter |
| | <220> |
| | <221> promoter |
| | <222> (385) . . . (666) |
| | <223> CB promoter |
| | <220> |
| | <221> TATA_signal |
| | <222> (639) . . . (642) |
| | <223> TATA signal |
| | <220> |
| | <221> Intron |
| | <222> (761) . . . (1733) |
| | <223> chicken beta actin intron |
| | <220> |
| | <221> misc_feature |
| | <222> (1745) . . . (2404) |
| | <223> HPRT |
| | <220> |
| | <221> polyA_signal |
| | <222> (2465) . . . (2591) |
| | <223> Rabbit beta globin polyA |
| 15 | <223> cytomegalovirus immediate early |
| | (CMV IE) enhancer |
| 16 | <223> chicken beta-actin promoter |

All publications cited in this specification are incorporated herein by reference in their entireties, as is the Sequence Listing filed herewith, labelled “21-9749PCT_Sequences_ST25”, and the sequences and text therein are incorporated by reference in their entireties. U.S. Provisional Patent Application No. 63/208,280, filed Jun. 8, 2021, and U.S. Provisional Patent Application No. 63/341,699, filed May 13, 2022 are incorporated herein by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising an adeno-associated virus (AAV) capsid and packaged therein a vector genome, wherein the vector genome comprises:
(a) an AAV 5′ inverted terminal repeat (ITR),
(b) an expression cassette comprising a coding sequence for a hypoxanthine-guanine phosphoribosyltransferase (HPRT) having a nucleic acid sequence of SEQ ID NO: 3 or a sequence at least 80% identical to SEQ ID NO: 3 which encodes amino acid sequence of SEQ ID NO: 4, which coding sequence is operably linked to expression control sequences which direct expression of the HPRT, and
(c) an AAV 3′ ITR.

2. The rAAV according to claim 1, wherein the AAV capsid is suitable for targeting to dopaminergic neurons.

3. The rAAV according to claim 2, wherein the AAV capsid is capable of targeting cells in the substantia nigra and/or ventral tegmental area.

4. The rAAV of claim 1, wherein the AAV capsid is a Clade F AAV.

5. The rAAV according to claim 4, wherein the AAV capsid is an AAVhu68 capsid.

6. The rAAV of claim 1, wherein the vector genome comprises the AAV 5′ ITR, a promoter, an enhancer, an intron, the HPRT coding sequence, a polyadenylation (polyA) signal, and the AAV 3′ ITR.

7. The rAAV according to claim 6, wherein the promoter is a CB7 promoter, a chicken beta-actin promoter or a tyrosine hydroxylase promoter.

8. The rAAV of claim 6, wherein the intron is a chicken beta-actin intron.

9. The rAAV of claim 6, wherein the polyA is a rabbit beta-globin (RBG) polyA.

10. The rAAV of claim 6, wherein the vector genome comprises the sequence of SEQ ID NO: 14.

11. The rAAV of claim 6, wherein the vector genome further comprises a spacer sequence of about 66 nucleotides between the AAV 5′ ITR and the promoter.

12. The rAAV of claim 6, wherein the vector genome further comprises a spacer sequence of about 88 nucleotides between the polyA and the AAV 3′ ITR and optionally has a sequence of nucleotide 1 to nucleotide 3006 of SEQ ID NO: 1.

13. The rAAV according to claim 12, wherein the AAV capsid is an AAVhu68 capsid.

14. A composition comprising a pharmaceutically acceptable aqueous liquid and a population of rAAV as measured in genome copies (GC) of claim 1.

15-16. (canceled)

17. A method of treating Lesch-Nyhan disease comprising direct delivery of a therapeutic gene for Lesch-Nyhan disease to the dopaminergic neurons of a patient in need thereof.

18. The method according to claim 17, wherein the delivery comprises injection into the substantia nigra and/or ventral tegmental area.

19. The method according to claim 17, further comprises using guiding the injection via magnetic resonance imaging (MRI).

20. A method of treating Lesch-Nyhan disease and/or a disorder associated with a deficiency in hypoxanthine-guanine phosphoribosyltransferase (HPRT) enzyme levels comprising direct delivery of a therapeutic gene for Lesch-Nyhan disease to the substantia nigra and/or ventral tegmental area of a patient in need thereof.

21. The method according to claim 20, further comprises guiding the injection via magnetic resonance imaging (MRI).

22. The method according to claim 17, wherein the therapeutic gene is hypoxanthine-guanine phosphoribosyltransferase (HPRT).

23. The method according to claim 20, wherein the therapeutic gene is hypoxanthine-guanine phosphoribosyltransferase (HPRT).

24. The method according to claim 17, wherein a rAAV comprising the therapeutic gene is delivered to the patient.

25. The method according to claim 24, wherein the rAAV comprises: (i) an adeno-associated virus (AAV) capsid; and (ii) a vector genome comprising: an AAV 5′ inverted terminal repeat (ITR), a coding sequence for HPRT operably linked to expression control sequences which direct expression of the HPRT, and an AAV 3′ ITR.

26. The method of treating according to claim 25, wherein a rAAV comprising the therapeutic gene is delivered to the patient, and wherein the rAAV is the rAAV according to claim 1.
